(19) United States
(12) Patent Application Publication
(10) Pub. No.: US 2023/0160009 A1
(43) Pub. Date: May 25, 2023

(54) PREDICTIVE RESPONSE BIOMARKER DISCOVERY PROCESS

(71) Applicant: HIFIBIO (HK) LIMITED, Central (CN)

(72) Inventors: Liang Schweizer, Cambridge, MA (US); Andreas Raue, Cambridge, MA (US); Dean Sung-Ling Lee, Cambridge, MA (US)

(73) Assignee: HIFIBIO (HK) LIMITED, Central, HK (CN)

(21) Appl. No.: 18/010,044

(22) PCT Filed: Jun. 17, 2021
(86) PCT No.: PCT/US2021/037916

§ 371 Date: Dec. 13, 2022

Related U.S. Application Data
(60) Provisional application No. 63/041,440, filed on Jun. 19, 2020.
(60) Provisional application No. 63/079,507, filed on Sep. 17, 2020.
(60) Provisional application No. 63/172,904, filed on Apr. 9, 2021.

(51) Int. Cl. C12Q 1/6881; C12Q 1/6886; C12N 5/00; C12N 5/0783

(52) U.S. Cl. CPC C12Q 1/6881; C12Q 1/6886; C12N 5/0012; C12N 5/0636; C12Q 2600/106; C12Q 2600/158

(57) ABSTRACT

The invention described herein provides a method to identify predictive response biomarkers (PRBs) for a treatment regimen. The PRBs can be used to identify suitable or unsuitable) patient population for the treatment regimen.

### REFERENCE TO RELATED APPLICATIONS

This application claims the benefit of the filing date of U.S. Provisional Patent Application Nos. 63/041,440, filed on Jun. 19, 2020; 63/079,507, filed on Sep. 17, 2020; and 63/172,904, filed on Apr. 9, 2021. The entire contents of the above-referenced applications, including any sequences in the listings and drawings, are incorporated herein by reference.

### BACKGROUND OF THE INVENTION

Most therapeutic interventions for a disease indication, such as a cancer, an autoimmune disease, or a neurodegenerative disease, are only effective in a portion of the patients, while being ineffective at best (if not harmful) to the remaining patients. Treating patients who may not benefit from a certain treatment is not only financially burdensome (especially for certain expensive treatment options involving proprietary innovative monoclonal antibodies) but may also be potentially harmful to the patients being treated, not to mention the lost opportunity for treating these patients with other, alternative treatments that may be effective.

Therefore, there is need to identify a proper patient population for a specific treatment regimen, in order to maximize the chance of successful treatment.

### SUMMARY OF THE INVENTION

One aspect of the invention provides a method of identifying a predictive response biomarker (PRB) for treating a disease (e.g., a cancer) using a T/B-cell-targeted immunomodulatory therapy, the method comprising the following steps: (a) in a pre-treatment sample of the disease (e.g., cancer), identifying response-capable T/B cells having TCR (T Cell Receptor)/BCR (B Cell Receptor) clonotypes identical to TCR/BCR clonotypes of clonally expanded T/B cells in a matching post-treatment sample of the disease (e.g., cancer), wherein said clonally expanded T/B cells in the matching post-treatment sample are clonally expanded following the treatment; and, (b) generating a list of genes upregulated and/or down-regulated in said response-capable T/B cells in the pre-treatment sample, to create the PRB; (c) optionally, each gene in said PRB is weighed using a logistic regression coefficient obtained by fitting said each gene as predictor with expansion status and/or clinical response status as outcome.

In certain embodiments, step (a) comprises: (1) generating said matching post-treatment sample by contacting an ex vivo culture of an untreated sample of the disease (e.g., cancer) with a T-cell-targeted immunomodulatory compound, under a condition and for a time period sufficient for an immunomodulatory effect of the compound on a T cell population within the ex vivo culture to manifest; (2) encapsulating individual T cells isolated, purified, or enriched from said ex vivo culture into picoliter droplets for single-cell profiling of a functional property, thereby separating each encapsulated individual T cells into a first pool of responder T cells and a second pool of non-responder T cells based on the presence or absence, respectively, of said functional property; (3) determining TCR clonotype for each encapsulated individual T cells in the first pool of responder T cells and the second pool of non-responder T cells, thereby identifying TCR clonotypes of the responder T cells as the TCR clonotypes of clonally expanded T cells in said matching post-treatment sample; (4) encapsulating individual T cells isolated, purified, or enriched from said pre-treatment sample into picoliter droplets to identify said response-capable T cells having TCR clonotypes identical to TCR clonotypes of said clonally expanded T cells in said matching post-treatment sample.

In certain embodiments, step (a) comprises: (1) obtaining, from a suitable donor, said pre-treatment sample and said matching post-treatment sample of the disease (e.g., cancer), wherein said matching post-treatment sample has been contacted with the compound, under a condition and for a time period sufficient for an immunomodulatory effect of the compound on a T cell population within the post-treatment sample to manifest; (2) encapsulating individual T cells isolated, purified, or enriched from said post-treatment sample into picoliter droplets for single-cell profiling of a functional property, thereby separating each encapsulated individual T cells into a first pool of responder T cells and a second pool of non-responder T cells based on the presence or absence, respectively, of said functional property; (3) determining TCR clonotype for each encapsulated individual T cells in the first pool of responder T cells and the second pool of non-responder T cells, thereby identifying TCR clonotypes of the responder T cells as the TCR clonotypes of clonally expanded T cells in said matching post-treatment sample; (4) encapsulating individual T cells isolated, purified, or enriched from said pre-treatment sample into picoliter droplets to identify said response-capable T cells having TCR clonotypes identical to TCR clonotypes of said clonally expanded T cells in said matching post-treatment sample.

In certain embodiments, step (b) comprises: (5) using single cell RNA sequencing (scRNA-seq) to identify said list of genes upregulated in said response-capable T cells in the pre-treatment sample, wherein each gene in said PRB has a log2-fold change of >0.2, and is expressed in <40% of T cells with TCR clonotypes that are not clonally expanded in the matching post-treatment sample.

In certain embodiments, said scRNA-seq is carried out using a gene panel specifically designed for a mechanism of action of the compound, or a pre-determined gene panel designed to assess immuno-modulation.

In certain embodiments, said pre-treatment sample is a blood sample.

In certain embodiments, the disease is a cancer, such as a solid tumor.

In certain embodiments, said ex vivo culture is freshly isolated from a disease tissue.

In certain embodiments, said ex vivo culture is established from a stored (e.g., a frozen) disease tissue.

In certain embodiments, the ex vivo culture is a single cell suspension.

In certain embodiments, the ex vivo culture is an adherent culture.

In certain embodiments, the time period is about 12-24 hrs, 24-36 hrs, 36-48 hours, up to 3 days, up to 4 day, or up to 5 days.

In certain embodiments, the condition comprises: one of a series of concentrations of the compound, and/or with or without combination with a second therapeutic agent.

In certain embodiments, the second therapeutic agent comprises a cytokine (e.g., IL-2, TNF), TCR stimulation (e.g., by CD3 cross-linking) and/or co-stimulation (e.g., CTLA, B7, CD28 etc).

In certain embodiments, the compound is an immuno-modulatory antibody.

In certain embodiments, step (2) is carried out by a microfluidic device.

In certain embodiments, said individual T cells are isolated, purified, or enriched by isolating, purifying, or enriching a specific T cell subset or population (such as CD4⁺ or CD8³⁰ T cells).

In certain embodiments, said individual T cells are isolated, purified, or enriched by generally isolating, purifying, or enriching for T cells.

In certain embodiments, said functional property comprises: IFN-γ secretion and/or upregulation of an activation marker.

In certain embodiments, said gene panel comprises a gene for proliferation (such as Ki67); a gene for stem-like feature (such as TCF7); a gene for T-cell activation (such as CD25, Granzyme B, Perforin, CD28, TNF, IL2, IFNG, 4-1BB, CD38, CD69, OX-40, GITR, IL4, IL6); a gene for T cell exhaustion (CD39, CTLA-4, EOMES, PD-1, TIGIT, 2B4, LAG-3, T-bet, TIM3, TOX, CD160, IL-10); a gene for migration (such as CD31, CD103, CXCR5, CXCR4, CCR7, CCR3, CCR4, CCR8, CCR5, CXCR3); and a combination thereof.

In certain embodiments, the gene panel further comprises CD4, CD8, FOXP3, CD62L, CD44, CD127, CD27, IL33R, and/or PTPRC.

In certain embodiments, in step (2), said function property is used to physically separating said responder T cells and non-responder T cells using a microfluidic device based on the presence or absence of said functional property.

In certain embodiments, in step (2), said function property is an activation marker or a response signature (either or both of which can be determined by, e.g., single-cell RNA sequencing), and wherein said responder T cells and non-responder T cells are not physically separated but are virtually distinguished based on the expression or lack of expression of said activation marker or response signature.

In certain embodiments, the method further comprises profiling the pre-treatment sample and/or the post-treatment sample to characterize the samples for, e.g., cell type composition (via, e.g., flow cytometry) and target expression on T cells.

Another aspect of the invention provides a method for selecting a patient for treatment of a disease (e.g., a cancer) using a T-cell-targeted immunomodulatory compound, said method comprising: obtaining a clinical response score for the patient, by assessing the expression status and/or expression level of genes in a predictive response biomarker (PRB) of the disease (e.g., cancer) obtained by the method of the invention, to determine whether said clinical response score for the patient exceeds a pre-determined threshold clinical response score;

wherein patients having clinical response scores exceeding the threshold score are identified as being beneficial for said treatment and are selected for said treatment, and/or,

wherein patients having clinical response scores below a second threshold score are identified as not being beneficial for said treatment and are not selected for said treatment.

Another aspect of the invention provides a method for treating a patient for a disease (e.g., a cancer) using a T-cell-targeted immunomodulatory compound, said method comprising: (i) obtaining a clinical response score for the patient, by assessing the expression status and/or expression level of genes in a predictive response biomarker (PRB) of the disease (e.g., cancer) obtained by the method of the invention, to determine whether said clinical response score for the patient exceeds a pre-determined threshold clinical response score; wherein patients having clinical response scores exceeding the threshold score are identified as being beneficial for said treatment and are selected for said treatment; and, (ii) administering a therapeutically effective amount of the compound to the patient identified in (i) as being beneficial for and being selected for said treatment.

It should be understood that any one embodiment of the invention, including those only described in the examples or claims, can be combined with any one or more embodiments of the invention, unless expressly disclaimed or being improper.

### BRIEF DESCRIPTION OF THE DRAWINGS

FIG. 1 is a non-limiting schematic drawing showing one embodiment of the invention.

FIG. 2 is a non-limiting schematic drawing showing another embodiment of the invention.

FIG. 3 is a list of exemplary genes in a pre-determined gene penal useful for profiling T cell response to treatment with an immunomodulatory, such as OX-40 agonists. The gene panel approach captures diverse T cell phenotypes while facilitating high-throughput screening.

FIG. 4 is a volcano plot showing differentially expressed (either significantly increased or significantly decreased) genes in expanded CD8⁺ T cells vs. non-expanded CD8⁺ T cells, based on comparison of single-cell RNA sequencing (scRNA-seq) data.

FIG. 5A shows that, in the atezolizumab arm of the POPLAR trial, survival analysis showed that the 47 patients who are CD8 T cell expansion signature high (Sig^{Hi}) responded better than the 46 patients who are signature low (Sig^{Lo}). The patients were split into two groups using a median cutoff value based on their signature scores.

FIG. 5B shows that, in the atezolizumab arm of the IMvigor210 trial, survival analysis showed that the 174 patients who are CD8 T cell expansion signature high (Sig^{Hi}) responded better than the 174 patients who are signature low (Sig^{Lo}). The patients were split into two groups using a median cutoff value based on their signature scores.

FIG. 6 shows the classification of expanded and non-expanded T cell subsets from patients treated with ICB. 12,551 T cells from BCC (a, c, e) and 14,522 T cells from SCC (b, d, f) were clustered and visualized with dimension reduction algorithms based on the similarity of their gene expression profiles. Association of single-cells with CD8+, CD4+ or regulatory T cell subsets was identified based on marker gene expression (a, b), and association of single-cells with patient identifiers is indicated (c, d). T cells that expanded in response to anti-PD-L1 treatment were identified based on scTCR-seq data and are marked in red. All other T cells are displayed in gray (e, f).

FIG. 7 shows differentially expressed genes that distinguish T cells that will expand after PD-1 blockade based on their pretreatment gene expression profile. Samples are divided into BCC (a, b) and SCC (c, d), and CD8+ (a, c) and CD4+ T cells (b, d). To select for the most clearly defined differentially expressed genes, we removed genes that have a high background expression, pass threshold for both fold-change and statistical significance (gray lines). The resulting list of differentially expressed genes are named and highlighted in color, where blue means downregulation on the expanded fraction, and red mean upregulation in the expanded fraction.

FIG. 8 shows an illustration of the score values of the identified predictive gene expression signatures as overlay on the single-cell data visualization. Two distinct signatures were identified, a CD8+ T cell signature in BCC patient samples that was a positive predictor of expansion (a), and a CD4+ T cell signature in SCC patient samples that was negative predictor of expansion (b). Signature score values are relative, unitless and indicated by a color scale with the score values shown in red that correlated with high likelihood of expansion and blue color indicating lower likelihood of expanding.

FIG. 9 shows a survival analysis of clinical trial data using the predictive signatures. Kaplan-Meier plots of progression-free survival (PFS) are shown for the atezolizumab treated patients in each clinical trial, with patients divided by their predictive signature scores above (Sig+) or below (Sig−) the median among all patients in the corresponding clinical trial. Censored observations are indicated by a plus symbol. Hazard ratios from a Cox proportional-hazard model analysis are shown (* pval<0.05, ** pval<0.01, *** pval<0.001). In b, patients were divided into four groups (low-low, low-high, high-low, and high-high) based on the two signatures shown in a, with the CD8 signature indicated first. Hazard ratios and significance relative to low-low groups are indicated.

FIG. 10 shows the results from logistic regression model training for deriving gene expression signatures that can optimally predict T cell expansion after PD-1 blockade. The figure show the results from training logistic regression models to predict the T cell expansion after PD-1 blockade at the single-cell level. For each of the T cell subsets independent signature have been derived, based on the analysis differential gene expression. For each subset, the threshold for differential gene expression fold-change was varied to investigated whether more stringent cut-offs would impact the predictive performance. The predictive performance was measured by accuracy (proportion of correct predictions, both true positives and true negatives, among the total number of cases), sensitivity (true positive rate), and specificity (true negative rate).

FIG. 11 shows the comparison of hazard-ratios between experimental and comparator arms in both biomarker positive (BM+) and negative (BM−) populations. Hazard ratios and two-sided statistical test from a Cox proportional-hazards model on patients in both groups are shown with significance indicated (* pval<0.05, ** pval<0.01, *** pval<0.001). † pval of 0.06.

FIG. 12 shows T cell subsets from pretreatment BCC and SCC samples with identification of patient of origin and expansion status. Cells were clustered and visualized with dimension reductions algorithms based on the similarity of their gene expression profiles. Association of single-cells with patient identifiers was indicated (a-d). T cells that have expanded in response to anti-PD-L1 treatment have been identified based on scTCR-seq data and are marked in red, all other T cells are displayed in gray (e-h).

FIG. 13 shows the workflow for predictive gene expression signature inference from single-cell data in a process diagram illustrating the steps in the workflow for predictive gene expression signature inference from scRNA-seq data.

FIG. 14 shows the workflow for application of predictive gene expression signatures to bulk RNA-seq data from clinical trials in a process diagram illustrating the steps in the workflow for application of the predictive gene expression signatures to bulk RNA-seq data from clinical trials.

FIG. 15 shows gene expression of FOXP3 (a) and GZMA (b) contained in the CD4− non-expansion signature identified from CD4+ T cells in SCC patient samples. Strength of expression of each gene is indicated by a color scale. The cluster identified as Tregs is indicate by a dashed circle line.

FIG. 16 shows a comparison of hazard-ratios between biomarker positive and negative populations. Hazard ratios and two-sided statistical test from a Cox proportional-hazards model on patients in both groups are shown with significance indicated (* pval<0.05, ** pval<0.01, *** pval<0.001).

### DETAILED DESCRIPTION OF THE INVENTION

In one aspect, the invention described herein provides a method to predict clinical response to a treatment, such as an anti-cancer T-cell based immunotherapy based on checkpoint blockade (e.g., anti-PD-L1 or anti-PD-1 treatment), based on the difference in gene expression profiles between T cell clonotypes in the tumor that eventually do expand after the immunotherapy, and T cell clonotypes in the tumor that eventually do not expand after the immunotherapy.

One salient feature of the invention is that the a gene signature, or a collection of predictive response biomarkers (PRBs), is identified in a population of untreated effector cells, such as T-/B-cells, partly based on single-cell profiling.

One advantage of single-cell profiling is that single-cell resolution increases statistical power, by leveraging heterogeneity within a patient's sample, which typically contains at least millions, if not tens of millions of heterogeneous cells, including disease (e.g., cancer) cells, immune cells (T cells, B Cells, NK cells, macrophages, neutrophils, etc). The complexity of the disease tissue and the microenvironment of the disease (such as Tumor Microenvironment or TME) is at least partially preserved during treatment; meanwhile, specific responses in individual target cells, such as immune cells as the target of an immunomodulatory drug (e.g., an immune checkpoint inhibitor), can be individually assessed.

A direct benefit of the increased statistical power is that the number of patients needed for the discovery of PRB s is significantly reduced.

In certain embodiments, the sample suitable for the method of the invention (i.e., to identify PRBs, and to treat patients based on the PRBs) can be an easily obtainable bodily fluid, such as a blood sample, which further enhances the ease with which the methods of the invention can be performed. In addition, trafficking of the target cells (e.g., immune cells) between the TME and the PBMC compartments enables possible blood-based biomarker screen to enhance clinical success.

Another distinguishing feature of the invention is that the PRBs are identified from untreated samples, which facilitates the discovery of biomarker that would otherwise not be identified based on traditional approaches that typically rely on expression or activity differences in treated samples compared to control.

In terms of using the PRBs identified using the methods of the invention, based on results from single-cell analysis, the identified PRBs can be implemented in patient stratification using more accessible technologies, such as immunohistochemistry (IHC), bulk qPCR, or gene panel.

The invention described herein is particularly suitable to identify PRBs in immune cells, such as T cells or B cells, although the same process can be readily adapted to other target cells or effector cells of a treatment.

Thus in one aspect, the invention provides a method of identifying a predictive response biomarker (PRB) for treating a disease or condition (e.g., a cancer) using a T/B-cell-targeted immunomodulatory therapy, the method comprising the following steps: (a) in a pre-treatment sample of the disease (e.g., cancer, autoimmune disease, or neurodegenerative disease), identifying response-capable T/B cells having TCR (T Cell Receptor)/BCR (B Cell Receptor) clonotypes identical to TCR/BCR clonotypes of clonally expanded T/B cells in a matching post-treatment sample of the disease (e.g., cancer, autoimmune disease, or neurodegenerative disease), wherein said clonally expanded T/B cells in the matching post-treatment sample are clonally expanded following the treatment; and, (b) generating a list of genes upregulated and/or down-regulated in said response-capable T/B cells in the pre-treatment sample, to create the PRB; (c) optionally, each gene in said PRB is weighed using a logistic regression coefficient obtained by fitting said each gene as predictor with expansion status and/or clinical response status as outcome.

As used herein, “predictive response biomarker (PRB)” includes genes the expression or the lack of expression of which can be used to predict whether a target cell (such as a T or B cell in an immunomodulatory therapy) or a patient comprising the target cell will respond to a specific treatment. The PRB may consists of one gene, or may comprise a collection of genes, which collection of genes may be collectively referred to as a “gene signature.”

The disease or condition may include any disease or condition that can be treated by an immunomodulatory treatment, such as a small molecule compound or an antibody (e.g., anti-PD-1 Ab or anti-PD-L1 Ab). For example, the disease can be a cancer, such as those cancers in the examples, including BCC, SCC, NSCLC, RCC, metastatic urothelial cancer, etc.

“Clonotype” as used herein refers to the specific pairing of BCR or TCR chains on B and T cells, respectively, since each mature B and T cell expresses on their surface a BCR or TCR that can potentially recognize an antigen. Such B and T cells can undergo clonal expansion under the right conditions, and upon binding to an antigen, and all B and T cells that are the progeny of such clonal expansion share the same BCR/TCR pairing that distinguishes them from BCR/TCR on the other B/T cells.

“Logistic regression” is a statistical model that in its basic form uses a logistic function to model a binary dependent variable. In regression analysis, logistic regression is estimating the parameters of a logistic model which is a form of binary regression. Mathematically, a binary logistic model has a dependent variable with two possible values, labeled “0” and “1.”

In certain embodiments, step (a) comprises: (1) generating said matching post-treatment sample by contacting an ex vivo culture of an untreated sample of the disease (e.g., cancer) with a T-cell-targeted immunomodulatory compound, under a condition and for a time period sufficient for an immunomodulatory effect of the compound on a T cell population within the ex vivo culture to manifest; (2) encapsulating individual T cells isolated, purified, or enriched from said ex vivo culture into picoliter droplets for single-cell profiling of a functional property, thereby separating each encapsulated individual T cells into a first pool of responder T cells and a second pool of non-responder T cells based on the presence or absence, respectively, of said functional property; (3) determining TCR clonotype for each encapsulated individual T cells in the first pool of responder T cells and the second pool of non-responder T cells, thereby identifying TCR clonotypes of the responder T cells as the TCR clonotypes of clonally expanded T cells in said matching post-treatment sample; (4) encapsulating individual T cells isolated, purified, or enriched from said pre-treatment sample into picoliter droplets to identify said response-capable T cells having TCR clonotypes identical to TCR clonotypes of said clonally expanded T cells in said matching post-treatment sample.

The T cell population may include all T cells, or subsets of T cells, such as helper CD4⁺ T cells (including Th1, Th2, Th9, Th17, and Tfh cells), cytotoxic CD8⁺ T cells (Tc cells, CTLs, T-killer cells, killer T cells), memory T cells (T_{CM} cells, T_{EM} cells and T_{EMRA} cells, T_{RM} cells, and virtual memory T cells), regulatory CD4⁺ T cells (FOXP3⁺ Treg cells and FOXP3⁻ Treg cells), Natural killer T cell (NKT), γδ T cells, which all can be selected based on the characteristic cell surface markers, cytokines secreted, and/or key transcription factors.

Different target cells (e.g., T cell subpopulations) may give rise to different PRBs for the same disease or indication.

In certain other embodiments, step (a) comprises: (1) obtaining, from a suitable donor, said pre-treatment sample and said matching post-treatment sample of the disease (e.g., cancer), wherein said matching post-treatment sample has been contacted with the compound, under a condition and for a time period sufficient for an immunomodulatory effect of the compound on a T cell population within the post-treatment sample to manifest; (2) encapsulating individual T cells isolated, purified, or enriched from said post-treatment sample into picoliter droplets for single-cell profiling of a functional property, thereby separating each encapsulated individual T cells into a first pool of responder T cells and a second pool of non-responder T cells based on the presence or absence, respectively, of said functional property; (3) determining TCR clonotype for each encapsulated individual T cells in the first pool of responder T cells and the second pool of non-responder T cells, thereby identifying TCR clonotypes of the responder T cells as the TCR clonotypes of clonally expanded T cells in said matching post-treatment sample; (4) encapsulating individual T cells isolated, purified, or enriched from said pre-treatment sample into picoliter droplets to identify said response-capable T cells having TCR clonotypes identical to TCR clonotypes of said clonally expanded T cells in said matching post-treatment sample.

Numerous microfluidic devices can be used to generate the picoliter droplets to encapsulate the cells for single cell profiling. See, for example, Gérard et al., Nat Biotechnol 38: 715-721, 2020 (incorporated by reference).

In certain embodiments, step (b) comprises: (5) using single cell RNA sequencing (scRNA-seq) to identify said list of genes upregulated in said response-capable T cells in the pre-treatment sample, wherein each gene in said PRB has a log2-fold change of >0.2, and is expressed in <40% of T cells with TCR clonotypes that are not clonally expanded in the matching post-treatment sample.

Single-cell transcriptome sequencing (or scRNA-seq for short) provides the expression profiles of individual cells. Compared to standard methods such as microarrays and bulk RNA-seq analysis to analyze the expression of RNAs from large populations of cells, which may obscure critical differences between individual cells within these populations, scRNA-seq can identify patterns of gene expression through gene clustering analyses. See Eberwine et al., Nature Methods. 11 (1): 25-27, 2014 (incorporated by reference).

In certain embodiments, the PRB is associated with a positive treatment outcome (e.g., treatment is predicted to be favorable or successful in the presence of the PRB). For example, in the example section below, it was shown that PRBs associated with the clonally expanded activated CD8⁺ T cells predict positive clinical outcome (e.g., longer progression-free survival).

In certain embodiments, the PRB is associated with a negative treatment outcome (e.g., treatment is predicted to be unfavorable or unsuccessful in the presence of the PRB). For example, in the example section below, it was shown that PRBs associated with the clonally expanded activated CD4⁺ T cells predict negative clinical outcome (e.g., shorter progression-free survival).

In certain embodiments, the scRNA-seq is carried out using a gene panel specifically designed for a mechanism of action of the compound, or a pre-determined gene panel designed to assess immuno-modulation.

In certain embodiments, the gene penal comprises genes representing T cell activation, exhaustion, migration, and/or proliferation.

In certain embodiments, the pre-treatment sample is a blood sample.

In certain embodiments, the disease is a cancer, such as a solid tumor.

In certain embodiments, the ex vivo culture is freshly isolated from a disease tissue.

In certain embodiments, the ex vivo culture is established from a stored (e.g., a frozen) disease tissue.

In certain embodiments, the ex vivo culture is a single cell suspension.

In certain embodiments, the ex vivo culture is an adherent culture.

In certain embodiments, the time period is about 12-24 hrs, 24-36 hrs, 36-48 hours, up to 3 days, up to 4 day, or up to 5 days.

In certain embodiments, the condition comprises: one of a series of concentrations of the compound, and/or with or without combination with a second therapeutic agent.

In certain embodiments, the second therapeutic agent comprises a cytokine (e.g., IL-2, TNF), TCR stimulation (e.g., by CD3 cross-linking) and/or co-stimulation (e.g., CTLA, B7, CD28 etc).

In certain embodiments, the compound is an immuno-modulatory antibody.

In certain embodiments, step (2) is carried out by a microfluidic device.

In certain embodiments, the individual T cells are isolated, purified, or enriched by isolating, purifying, or enriching a specific T cell subset or population (such as CD4⁺ or CD8⁺ T cells).

In certain embodiments, the individual T cells are isolated, purified, or enriched by generally isolating, purifying, or enriching for T cells.

In certain embodiments, the functional property comprises: secretion of a cytokine (e.g., IFN-γ), and/or upregulation of an activation marker.

In certain embodiments, the gene panel comprises a gene for proliferation (such as Ki67); a gene for stem-like feature (such as TCF7); a gene for T-cell activation (such as CD25, Granzyme B, Perform, CD28, TNF, IL2, IFNG, 4-1BB, CD38, CD69, OX-40, GITR, IL4, IL6); a gene for T cell exhaustion (CD39, CTLA-4, EOMES, PD-1, TIGIT, 2B4, LAG-3, T-bet, TIM3, TOX, CD160, IL-10); a gene for migration (such as CD31, CD103, CXCR5, CXCR4, CCR7, CCR3, CCR4, CCR8, CCR5, CXCR3); and a combination thereof.

In certain embodiments, the gene panel further comprises CD4, CD8, FOXP3, CD62L, CD44, CD127, CD27, IL33R, and/or PTPRC.

In certain embodiments, in step (2), said function property is used to physically separating said responder T cells and non-responder T cells using a microfluidic device based on the presence or absence of said functional property.

In certain embodiments, in step (2), said function property is an activation marker or a response signature (either or both of which can be determined by, e.g., single-cell RNA sequencing), and wherein said responder T cells and non-responder T cells are not physically separated but are virtually distinguished based on the expression or lack of expression of said activation marker or response signature.

In certain embodiments, the method further comprises profiling the pre-treatment sample and/or the post-treatment sample to characterize the samples for, e.g., cell type composition (via, e.g., flow cytometry) and target expression on T cells.

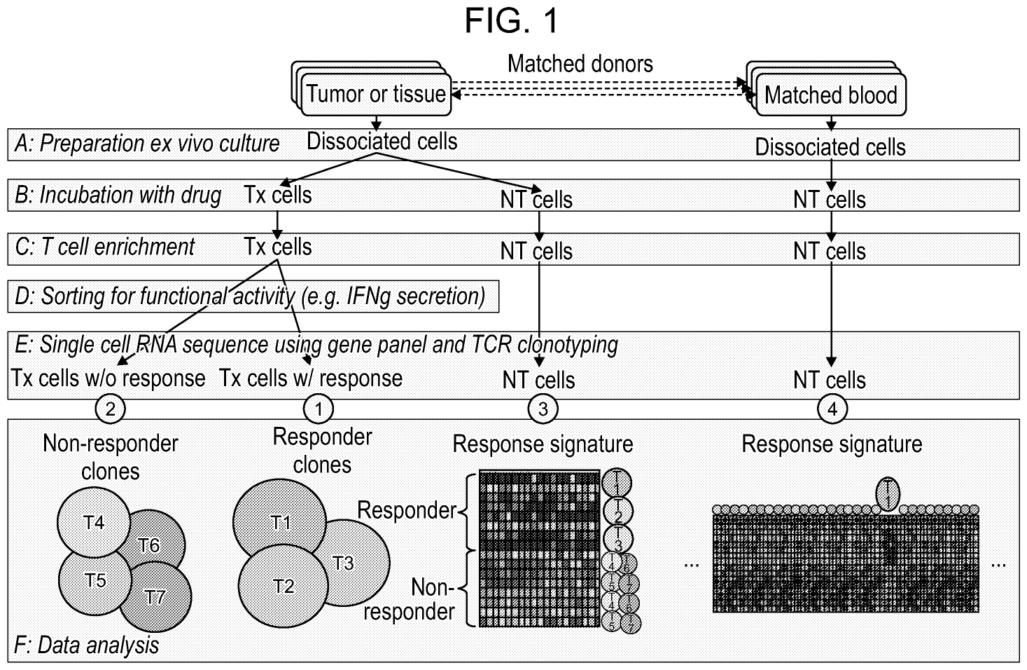

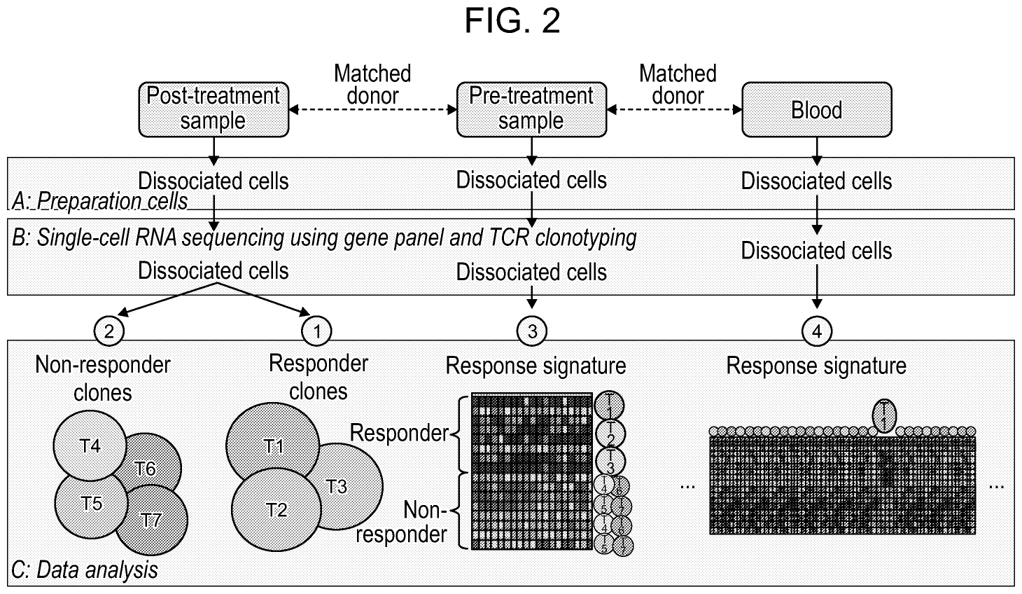

The invention described herein may be better illustrated in view of the following descriptions of the non-limiting, exemplary embodiments depicted in the schematic drawings in FIGS. 1 and 2.

FIG. 1 depicts an exemplary embodiment in which fresh, untreated patient sample for a disease, such as a cancer, is obtained for the purpose of identifying the predictive response biomarkers (PRBs) that may be useful to stratify patients for a particular treatment to ensure maximum chance of therapeutic efficacy. The example focuses on T cells as the target cells for a potentially immunomodulatory drug (e.g., an anti-PD-1 or anti-PD-L1 antibody), although the same approach can be applied to other cells, such as B cells. For the sake of simplicity, the description is focused on T cells, which is not limiting.

Specifically, FIG. 1 illustrates an exemplary procedure that can be used to identify predictive response biomarkers (PRBs) for a T-cell targeted immuno-modulatory drug, such as an agonist antibody specific for an immune-modulatory receptor expressed on the surface of a target T cell (e.g., OX40), using ex vivo cell culture, single-cell screening, and matched TCR clonotyping and phenotyping.

The process starts with tissue collected from cancer patients with accessible solid tumor lesions, for instance by resection or biopsy, before entering the experiment workflow (A-F).

In (A) Preparation of ex vivo culture: The fresh tissue is dissociated into a single cell suspension, which can be optionally further characterized using, for example, cell composition profiling with flow cytometry, in order to characterize the cell type composition of the sample. Target expression (e.g., OX40 receptor expression) on T cells can also be assessed at this stage. Several aliquots of the sample can optionally be frozen and stored for repeated experiments in the future. The remaining dissociated cells can then be distributed across the one or more experimental conditions.

(B) More than one experimental conditions can be tested, either sequentially or in parallel. Experimental conditions may include control or non-treated conditions (NT), for instance, by using an isotype-matched control for any antibody-based treatment condition. Experimental conditions can also include treatment conditions (Tx) using immuno-modulatory drugs, for instance an immuno-modulatory antibody. This can include different concentrations, alone or in combination with other drugs, or T cell-relevant supplementary conditions such as cytokines (e.g., IL-2, TNF), TCR stimulation (e.g., using anti-CD3 crosslinking), or co-stimulation (e.g., CD28 stimulation). Regardless of the specific conditions used for a particular treatment, the ex vivo cell cultures are incubated with the chosen treatment conditions for enough time in order to let the treatment act on the target cells (T cells) before activity readouts on single cells are assessed. For gene expression or early activation markers, for example, typical incubation times can range from 12-48 hours, but cells can be cultured for up to 3-5 days for a typical phenotypic readouts to manifest.

(C) T cell enrichment: After enough time has passed for the immuno-modulatory drug (e.g., anti-OX40 agonist antibodies) to induce activity on target cells (T cells) via their mechanism of action, for each experimental condition, target cells are enriched from the heterogeneous population of cells in the ex vivo cell culture for further characterization at the single cell level. The cells can be enriched either broadly for any and all T cells, or for specific T cell subsets, such as CD4⁺ or CD8⁺ T cells. The subpopulations can be analyzed separately, and the results can be considered together. Regardless of the enrichment chosen, the enriched T cells then enter single-cell profiling.

(D) For each treatment (Tx) condition, single enriched target cells (T cells) can be encapsulated in picoliter droplets using microfluidic devices, and each assayed for one or more functional properties, such as IFN-γ secretion, or activation marker(s) up-regulation. Based on the presence or absence of such functional properties, the target cells (T cells) can be physically or virtually sorted into a first pool of responders (1) (Tx cells w/ response) and a second pool of non-responders (2) (Tx cells w/o response) to the specific treatment condition. Meanwhile, the non-treated (NT) condition sample can be similarly enriched for the same target cells (e.g., T cells), and similarly encapsulated in picoliter droplets using microfluidic devices (3).

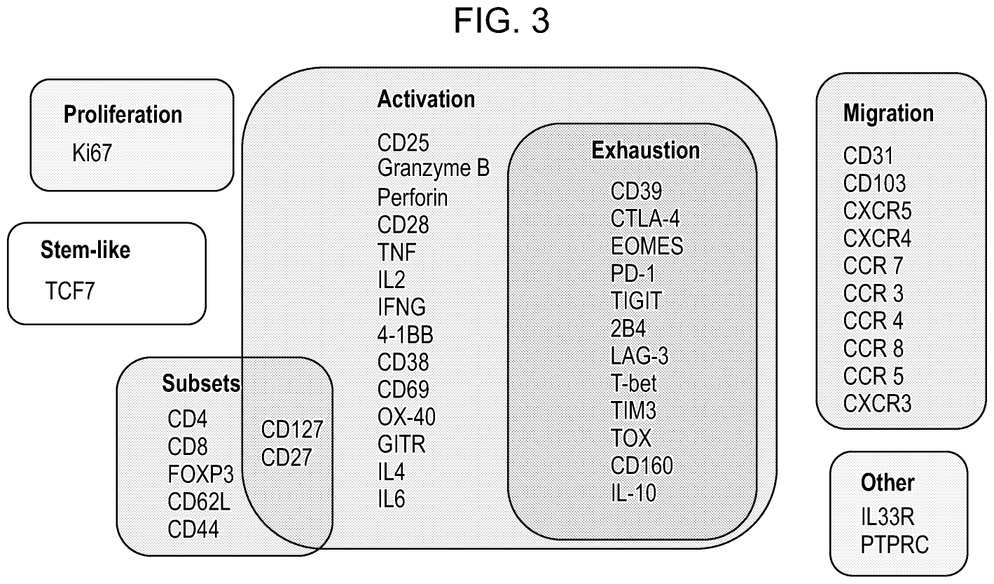

(E) TCR clonotyping and/or Single-cell RNA sequencing (e.g., using gene panel): Each single target cell (T cell) in the Tx conditions, including the physically or virtually sorted fractions (1) and (2), as well as the NT condition (3), can now be subject to TCR clonotyping to identify their respective association with a clonotype. Meanwhile, each single target cell (T cell), especially those in NT condition (3), can be profiled with single-cell RNA sequencing, optionally by using a gene panel specifically designed for the respective mechanism of action of the drug under investigation (such as the anti-OX40 agonist antibody). An exemplary gene panel for OX40 agonist antibody as the immune-modulatory drug is shown in FIG. 3. Thus each TCR clonotype in the NT condition (3) is associated with an expression profile in terms of the gene panel (or the whole genome expression profile if gene panel is not used).

(F) Data analysis. Solid tumors are known to contain clonally expanded T cells that are associated with anti-tumor antigen specificity, and that these expanded T cell clonotypes are believed to be the important cells that are responsible for anti-tumor activity. Such T cells, however, are frequently found to be inhibited in the tumor microenvironment, possibly due to checkpoint inhibition through, for example, the PD-1/PD-L1 pathway, due to the presence of PD-L1 expressed by the tumor cells. Thus immune-checking blocker (such as anti-PD-1 antibody or anti-PD-L1 antibody), or other immune-modulatory drug (such as anti-OX40 agonist antibody) may be used to relieve such inhibition to re-stimulate the dormant T cells.

While not wishing to be limited to any particular theory, it is believed that, for the purpose of identifying PRBs, the expanded T cell clonotypes (as illustrated by T1-T3 in FIG. 1) in the responder fraction (1) are the important drivers of anti-tumor activity as consequence of the drug treatment. On the other hand, however, there will be non-responder T cell clonotypes (as illustrated by T4-T7 in FIG. 1) that will be collected in the non-responder fraction (2). By comparing clonotypes in fractions (1) and (2), clonotypes that eventually did respond to the drug treatment (such as those in T1-T3) and clonotypes that eventually did not respond to the drug treatment (such as those in T4-T7) can be identified/assigned.

Importantly, since all T cells of all clonotypes were randomly distributed into the Tx and NT experimental conditions in (A), the important responder clones will be expanded and be present in the original sample multiple times, including being present in the never treated sample under NT condition (3). The responders in NT (3) have the same clonotype of the responders eventually identified as responders in Tx (1), and the difference is that such responders in NT (3) have not previously been exposed to treatment condition Tx. Other clonotypes in NT (3) are presumably those of the non-responders.

Thus, according to the method of the invention, the responding and nonresponding clonotypes in the NT condition (3) can be identified, even though none of the T cells in NT condition have previously been exposed to the treatment condition Tx.

By comparing the gene expression profiles in (3), between cells with clonotypes associated with the responders to the drug treatment (illustrated by T1-T3) and cells with clonotypes associated with non-responders with respect to the drug treatment (illustrated by T4-T7), markers specific for responding T cell clonotypes, before they are treated by treatment condition Tx, can be identified as the PRBs (predictive response signature) for the drug treatment.

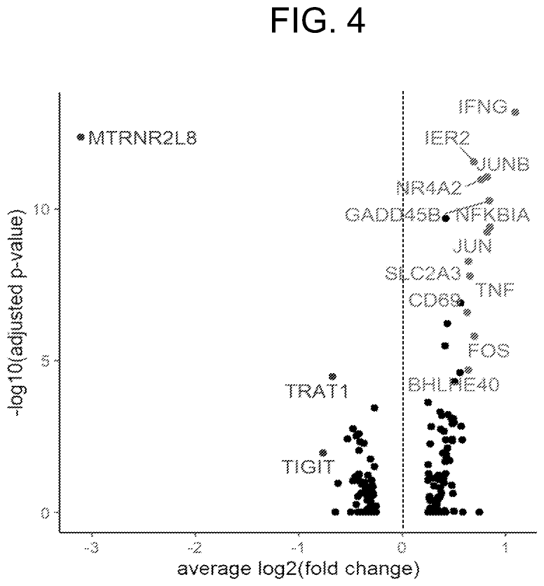

In certain embodiments, the PRB comprises one or more genes the expression of which is significantly increased or significantly decreased in the responding (e.g., T) cells compared to that in the non-responding (e.g., T) cells. See FIG. 4.

In certain embodiments, one or more (e.g., substantially all or all) genes in the PRB have a log2-fold change of >0.2, >0.3, >0.4, >0.5, >0.6, >0.7, >0.8, >0.9, >1 (2-fold) or more.

In certain embodiments, one or more (e.g., substantially all or all) genes in the PRB is/are expressed in <5%, 10%, 20%, 30%, <40%, <50%, <60% of T/B cells with TCR/BCR clonotypes that are not clonally expanded in the matching post-treatment sample.

In certain embodiments, all genes in the PRB have a log2-fold change of >0.2, and are expressed in <40% of T/B cells with TCR/BCR clonotypes that are not clonally expanded in the matching post-treatment sample.

In certain embodiments, the genes in the PRB are individually weighed based on the individual predictive value of the genes. For example, some genes differentially expressed (e.g., significantly increased or decreased in the responders compared to the non-responders, based on the threshold log2-fold value, and prevalence of expression % value in responders vs. non-responders) may be strongly correlated to a desired biological function or outcome, such as T cell expansion, or patient being responsive to a treatment. Such genes carry a high weight (e.g., close to 1 in a scale of 0 to 1, with 0 being no weight and 1 being the maximum weight). Conversely, some genes may have relatively low correlation with the desired biological function or outcome, such that increased expression of such genes are only correlated to, e.g., 30% of the favorable biological outcome. Such genes, though still part of the PRB, may be given a lower weight (e.g., 0.3 in a scale of 0-1). According to this weight assignment approach, genes for which an increase in expression is anti-correlated with the desired biological function or outcome are given a negative weights (e.g., −0.6). Such PRB with each or the majority of its genes weighed according to their respective predictive value is likely more accurate for prediction, compared to a corresponding PRB in which each gene is given equal weight, regardless of the individual genes' predictive value for the specific biological outcome.

The assignment of the weight to the genes in the PRB can be carried out as part of the machine learning process, such as machine learning based on logistic regression classifiers. In this embodiment, training datasets may be provided to include the identity of the genes in the PRB, and/or their respective expression levels (either as absolute value in the responders, or relative value compared to the non-responders), as well as the associated biological outcomes (e.g., clonal expansion or not for T/B cells having/not having such increased/ decreased gene expression, patient being responsive to treatment or not, etc). Given the numerous (e.g., tens of thousands, if not hundreds of thousands of) available T/B cells able to provide such training datasets, the weighing of the individual genes in the PRB and the power of prediction for the PRB can be highly accurate and statistically powerful.

Alternatively or in addition, cells obtained from matched blood samples from the same donor can be assayed using the same process. In the resulting data (4), clonotypes that do eventually respond to the drug treatment from the earlier steps can again be identified (illustrated by T1). Based on the corresponding gene expression signature in the blood (similar to the NT condition (3) above), the PRBs can be identified based on a blood sample, which enables a more practical implementation of the method of the invention, compared to a corresponding method using a tissue sample (such as a biopsy) from a disease tissue such as tumor.

As mentioned above, fractions (1) and (2) can be physically separated through using a microfluidic device with sorting capability, based on a pre-determined functional property. For example, the functional property can be the expression of a cytokine such as IL-12 or IFN-γ in T cells, or the secretion of an antibody by B cells, or the expression of a cell surface marker, all of which can be labeled by a fluorescent signal or dye that can be used for sorting.

Alternatively, no physical sorting is required, if, for example, such expressed markers are not easily detected or labeled for physical sorting. In this case, the responder fraction (1) and the non-responder fraction (2) can be virtually sorted based on the expression of certain genes or a panel of genes for the responders, and the lack of such expression or the expression level difference of such genes in the non-responders. This can be effected by using the same gene panel in the NT condition (3), or a different gene panel designed to best capture the difference between fractions (1) and (2) in gene expression.

In another embodiment, instead of using fresh disease tissues not previously treated by the treatment (e.g., a T-cell targeted immuno-modulatory drug) for testing the treatment in ex vivo culture, a treated (post-treatment) sample and a matching untreated (pre-treatment) sample can be used similarly, without the use of ex vivo culture. This can be particularly useful in cases when stored treated and matching untreated samples are available. FIG. 2 is an illustration of this exemplary embodiment, for identifying predictive response biomarkers (PRBs) for a T cell-targeted immuno-modulatory drug (such as anti-OX40 agonist antibody), using pre- and post-treatment patient samples, single-cell screening, and matched TCR clonotyping and phenotyping.

Similarly, this process starts with matched pre- and post-treatment tissues collected from patients with accessible lesions, such as solid tumor. Again, the samples can be obtained using any conventional means such as resection or biopsy, before the samples enter the experiment workflow (A-C).

(A) Preparation of cells: The pre- and post-treatment tissues can be dissociated into single cell suspensions. Several aliquots of these samples can be frozen and stored for repeated experiments in the future. Cells from each sample can be enriched, either broadly for T cells or specific T cells subsets (e.g., CD4⁺ or CD8⁺ T cells). The enriched T cells can then enter the next single-cell profiling step, by using, for example a microfluidic device that encapsulating individual target cells (e.g., T cells) into picoliter droplets. As before, the enriched T cells can be sorted virtually or physically based on the presence or absence of a functional property, to create a responder pool (1) and a non-responder pool (2). The pre-treatment sample gives rise to non-treatment condition NT (3).

(B) TCR clonotyping and/or Single-cell RNA sequencing (e.g., using gene panel): Cells from each sample can be profiled to identify TCR clonotypes for each single T cell, in order to identify their respective association with a clonotype. Additionally, cells of the samples, particularly cells of the pre-treatment sample (the NT (3) condition) can be profiled using single-cell RNA sequencing, either by using whole transcriptome sequencing, or by using a more focused gene panel specifically designed for the respective mechanism of action of the drug under investigation.

(C) Data analysis. As before, based on the data from the post-treatment sample, responder T cells (1) or non-responder T cells (2) can be identified based on the presence or absence of a functional property, such as their gene expression pattern, e.g., the expression of one or more activation markers (e.g. IL-2RA), cytokines (e.g. IFN-γ), and/or increased clonal expansion by comparing to the clonotyping of the pre-treatment sample.

Solid tumors contain clonally expanded T cells that are associated with anti-tumor antigen specificity, and these expanded T cell clonotypes are believed to be the important cells that are responsible for anti-tumor activity. Therefore, it is believed, for the purpose of the PRB identification approach, that the expanded clonotypes (illustrated by T1-T3) in the responder fraction (1) are the important drivers of anti-tumor activity as the consequence of the drug treatment. On the other hand, non-responder T cell clonotypes (illustrated by T4-T7) will be collected in the non-responder fraction (2).

Importantly, since all T cells of all clonotypes are initially randomly distributed into the Tx (post-treatment) and NT (matching pre-treatment) experimental conditions in (A), and important responder clones are expanded and are present in the original sample multiple times, the responding and nonresponding clonotypes can be identified from the pre-treatment sample (3). By comparing the gene expression profiles in (3), between clonotypes associated with responder to the drug treatment (illustrated by T1-T3) and clonotypes associated with non-responders to the drug treatment (illustrated by T4-T7), markers (PRBs) specific for responding T cell clonotypes can be identified, before the responders are subject to the treatment condition. The identified markers therefore constitute a predictive response biomarker (PRB) for the drug treatment, according to the method of the invention.

Alternatively or in addition, cells obtained from matching blood samples from the same donor can be assayed using the same process (FIG. 2). In the resulting data (4), clonotypes that do respond to the drug treatment from the earlier steps can again be identified (illustrated by T1). Based on the corresponding gene expression signature in the blood, it can be confirmed that the identified response signature (PRBs) can be based on a blood sample, which enables a more practical implementation of the subject PRBs for the drug treatment, compared to using a tissue sample from a tumor.

Although the above descriptions use T cell stimulation by an immunomodulatory drug as an illustrative example, one can readily envision that the methods of the invention can be applied to other immune cells, including B cells and innate immune system effector cells, and to other treatment conditions such as combination of different drugs with different mechanisms of actions, and/or different dosing regimens and treatment schedules.

Thus in a more general sense, the invention described herein provides a method of identifying PRBs in a pre-treatment sample from a patient with respect to a particular treatment, and a method of using PRBs so identified to predict the treatment outcome of for individual patients, such that patient populations can be properly and efficiently stratified for any specific treatment.

The method of the invention utilizes a single-cell based approach that starts with material/sample collection from patients at the disease site, for instance, solid tumor tissue from a cancer patient. Optionally, matching PBMCs from the same patients can also be collected to facilitate identification of potential blood-based biomarker. Next, the tissue can be processed for ex vivo culture and treatment with one or more experimental conditions/ drugs, either in parallel, or sequentially. At this stage, several pre-tests can optionally be performed in order to better characterize the samples. For instance, the tissue composition can be determined using flow cytometry to characterize the resident cell types within the tissue (e.g., T cells in a tumor tissue). Also, drug target expression on the target cells can be quantified, and experimental conditions can be optimized in this pre-test step. After the ex vivo cultures of the tissue samples have been treated with the experimental conditions/drugs, and after sufficient time has passed to induce any drug effects, the cell pool is harvested. At this point, conventional/bulk analysis can be used to characterize the treatment outcome, such as flow cytometry, qRT-PCR, or ELISA. Next, target cells (such as T or B cells) are purified, and enter the single-cell analysis pipeline. At this stage, all experimental conditions are profiled at the single-cell level, both for readouts that characterize the activity of the treatments but also to characterize the state of the cells in the control conditions. For instance, microfluidics systems can be used to screen for a functional activity readout of the treatments, such as the expression of activation markers or secreted soluble factors such as cytokines. In addition, sequencing based readouts such as TCR or BCR clonotyping and gene expression readouts can be employed to characterize activity and characteristics of cells. Finally, the obtained single-cell data from the different experimental conditions and readouts, as well as the data from the conventional analysis, enter the computational analysis to identify predictive response biomarkers (PRBs) based on the differences observed in the treated conditions compared to control.

Another aspect of the invention provides a method for selecting a patient for treatment of a disease (e.g., a cancer) using a T-cell-targeted immunomodulatory compound, said method comprising: obtaining a clinical response score for the patient, by assessing the expression status and/or expression level of genes in a predictive response biomarker (PRB) of the disease (e.g., cancer) obtained by the method of the invention described herein, to determine whether said clinical response score for the patient exceeds a pre-determined threshold clinical response score; wherein patients having clinical response scores exceeding the threshold score are identified as being beneficial for said treatment and are selected for said treatment; and/or wherein patients having clinical response scores below a second threshold score are identified as not being suitable for said treatment and are not selected for said treatment.

Yet another aspect of the invention provides a method for treating a patient for a disease (e.g., a cancer) using a T-cell-targeted immunomodulatory compound, said method comprising: (i) obtaining a clinical response score for the patient, by assessing the expression status and/or expression level of genes in a predictive response biomarker (PRB) of the disease (e.g., cancer) obtained by the method of any one of the method of the invention, to determine whether said clinical response score for the patient exceeds a pre-determined threshold clinical response score; wherein patients having clinical response scores exceeding the threshold score are identified as being beneficial for said treatment and are selected for said treatment; and, (ii) administering a therapeutically effective amount of the compound to the patient identified in (i) as being beneficial for and being selected for said treatment.

### EXAMPLES

### Example 1

The example described herein provides an illustration for one non-limiting embodiment of the invention, and is by no means limiting.

In one previous study, 4 cancer patients having SCC (squamous cell carcinoma) and 11 patients with BCC (basal cell carcinoma) were subjected to anti-PD-1 antibody therapy. About an average of 31 days post treatment for SCC patients, and about an average of 54 days post treatment for BCC patients, site-matched normal or untreated samples were obtained from the patients, and more than 26,000 T cells (TILs, tumor infiltrating lymphocytes) isolated from these samples were subjected to single cell profiling. Specifically, single-cell RNA sequencing (scRNA-seq) and single-cell TCR sequencing (scTCR-seq) were performed on isolated T cells from each sample to allow identification of TCR clonotypes that have expanded post-treatment. It was concluded that the expanded T cell clones consisted mainly of novel clonotypes that migrated from outside the tumor. The study did not examine any expanded T cell clones originated within the tumors.

Thus a question arises as to how the gene expression of the expanded TCR clonotypes differ from those of the non-expanded TCR clonotypes in the tumor, before checkpoint blockade by the anti-PD-1 antibody treatment. Further, how well do these gene expression differences can be used to predict clinical responses to the anti-PD-1 or anti-PD-L1 treatment.

This example demonstrates that, using TCR clonotypes as a matching condition, it is possible to identify T cells that expanded post-treatment, in the pre-treatment samples.

In general, for an immuno-modulatory drug targeting T cells (i.e., modulating T cell activity), T cells from BCC and SCC pre-treatment samples were enriched by specific T cell markers. In this example, the T cells were further split into CD8⁺ T cells from BCC/SCC pre-treatment samples, and CD4⁺ T cells from BCC/SCC pre-treatment samples. These pools of T cells were subject to the method of the invention to identify genes up-regulated in clonally expanded CD8/CD4 T cells. Then a gene expression filter was applied to identify a list of up-regulated genes (log2-fold-change>0.2) that are expressed in a low percentage (e.g., <40% in this case) of non-expanded T cells. This list constitutes a gene signature or predictive response biomarker (PRB) for the treatment. See FIG. 4.

Logistic regression was then performed with genes of the gene signature as predictors, and the T cell expansion status as the outcome. The coefficients so obtained were used as weights for each gene for future prediction. If the up-regulation of a gene is tightly correlated with the clonal expansion of T cells, the gene possesses higher predictive value or power over another gene that is less tightly correlated with the clonal expansion of T cells.

Next, bulk RNA-seq data were obtained from several anti-PD-L1 (atezolizumab) clinical trials, namely IMvigor210 (treating metastatic urothelial cancer, n=348), IMmotion150 (treating renal cell carcinoma or RCC, n=86 for atezolizumab only, n=88 for atezolizumab+bevacizumab (which is an anti-angiogenic drug designed to block VEGF)), and POPLAR (treating NSCLC, n=93). From these studies, bulk RNA-seq data was collected from tumor samples pre-treatment, and progression-free survival (PFS) data was collected as treatment endpoint.

With these data at hand, the PRBs (i.e., signatures of T cell expansion) can be correlated to actual clinical response or PFS data.

Specifically, the weights for each gene signature from the scRNA-seq data was applied to the corresponding genes in the clinical trials' bulk RNA-seq data, to calculate a score for each patient as the weighted sums of genes from a PRB/gene signature. Taking the median score of the patients, half of the patients with scores higher than the median were designated as the Sig^{high} group, and the other half of the patients with scores lower than the median were designated as the Sig^{low} group. The censored PFS data was then fitted to a Cox proportional-hazards model to obtain hazard ratios between the Sig^{high} and Sig^{low} groups, and Kaplan-Meier (KM) plots for progression-free survival (PFS). Here, HR is defined as the risk of outcome (e.g., death) in one group (e.g., the Sig^{high} group)/the risk of outcome (e.g., death) in another group (e.g., the Sig^{low} group), occurring at a given interval of time.

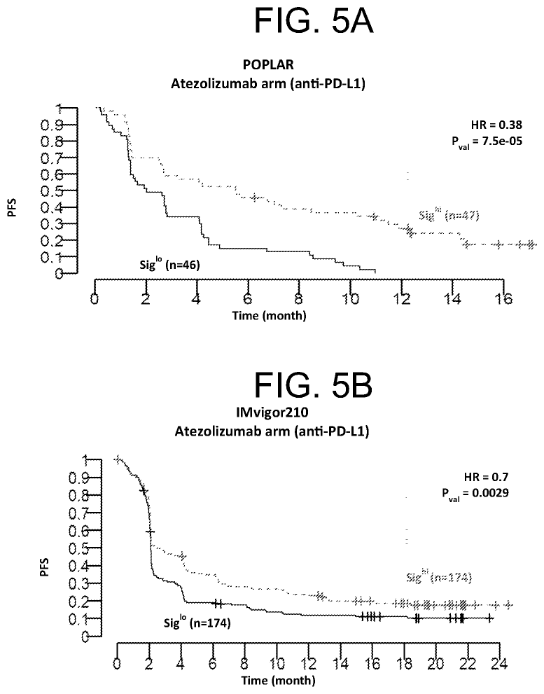

The results showed that a single CD8 T cell expansion signature (PRB) predicted positive clinical response across different trials. Specifically, in the IMvigor210 trial with 348 patients, applying the CD8⁺ PRB to the patient pre-treatment bulk RNA data, the hazard ratio (HR) for patients in the Sig^{high} group over the Sig^{low} group is 0.7 (p<0.0029), indicating that the Sig^{high} group of patients have significantly better chance of survival compared to the Sig^{low} group of patients. See FIG. 5B. Likewise, in the POPLAR trial with 93 patients, the HR for Sig^{high} group is 0.38 (p=7.5e−05) over the Sig^{low} group, indicating a statistically significant survival advantage. See FIG. 5A. This data demonstrates that the CD8⁺ T cell expansion signature is associated with a positive clinical response, and the predictive value of this signature outperforms that of a published signature (see comparison below).

| | CD8-expansion Signature | Wu et al., Nature (2020)* |
|---|---|---|
| POPLAR | HR = 0.38 | HR = 0.45 |
| IMvigor210 | HR = 0.70 | HR = 0.72 |

*Wu et al., Nature 579: 274-278 (2020).

Similar results were also obtained in the IMmotion 150 trial (atezolizumab alone) with 86 patients, with HR ratio being 0.87, though the p value(p=0.58) does not support statistical significance. In the same trial with atezolizumab+bevacizumab (n=88 patients), the HR is 0.68 (p=0.12).

On the other hand, data below shows that CD4 T cell expansion signatures predict negative clinical response, supporting the other embodiment of the invention in which clonal expansion by the CD4⁺ T cells appeared to be detrimental to survival in that treatment.

In particular, in the IMvigor210 trial, applying the PRB (GZMA) obtained from CD4³⁰ T cells resulted in an HR of 1.21 (p=0.11) for Sig^{high} patients, suggesting slightly lower survival compared to Sig^{low} patients.

In POPLAR trial with GZMA gene in the PRB, the HR was 1.44 (p=0.11).

In IMmotion150 trial with atezolizumab alone, the PRB (GZMA) resulted in an HR of 1.59 (p=0.059) for Sig^{high} patients. With atezolizumab and bevacizumab, the PRB (GZMA) produced an HR of 1.86 (p=0.013).

Overall, the data above shows that up-regulated genes in CD8 or CD4 T cells that expand after anti-PD1 treatment can predict a positive or a negative clinical response, respectively. Gene signatures or PRBs derived from one cancer (e.g., BCC and SCC datasets) applies to other tumor types (RCC, NSCLC, metastatic urothelial cancer). Thus, combined clonotype and phenotype analysis can be used to identify predictive response signatures that correlates with patient outcome. The methods described herein provide unbiased and systematic approach for the discovery of predictive response biomarkers.

### REFERENCES

1. clinicaltrials.gov/ct2/show/NCT02108652, “A Study of Atezolizumab in Participants With Locally Advanced or Metastatic Urothelial Bladder Cancer (Cohort 2).”
2. Balar et al., Atezolizumab as first-line treatment in cisplatin-ineligible patients with locally advanced and metastatic urothelial carcinoma: a single-arm, multicentre, phase 2 trial. The Lancet, 389(10064):67-76, 2017.
3. clinicaltrials.gov/ct2/show/NCT01903993, “A Randomized Phase 2 Study of Atezolizumab (an Engineered Anti-PDL1 Antibody) Compared With Docetaxel in Participants With Locally Advanced or Metastatic Non-Small Cell Lung Cancer Who Have Failed Platinum Therapy—‘TOPLAR’.”
4. Weinstock et al., U.S. Food and Drug Administration Approval Summary: Atezolizumab for Metastatic Non—Small Cell Lung Cancer. Clin Cancer Res. 23(16):OF1-OF6, 2017.

### Example 2

Single-Cell Immune Profiling Using Clonotype Barcoding Identifies Biomarker Signatures that Predict Response to Immune Checkpoint Blockade.

The identification of predictive biomarkers for patient treatment response is urgently needed to increase the probability of success of existing and novel experimental therapies. Single-cell profiling has provided novel biological insights into drug responses in the tumor microenvironment but its potential for biomarker discovery has not yet been explored for therapeutic purposes. This example describes a novel approach to discover predictive response biomarkers from single-cell data that uses the T cell receptor sequence intrinsic to each T cell to match clonotypes between pre- and post-treatment tumor samples. As a result, a predictive gene expression signature were identified from a small patient cohort, for immune checkpoint blockade, and its predictive performance was validated using data from three larger clinical studies.

The results presented herein demonstrated that applying clonotyping with genomic profiling is a novel approach using single-cell technology for biomarker identification. This approach leads to enhanced probability of success and reduced clinical trial size and therefore, significantly impact future clinical developments of novel immunomodulatory therapeutics.

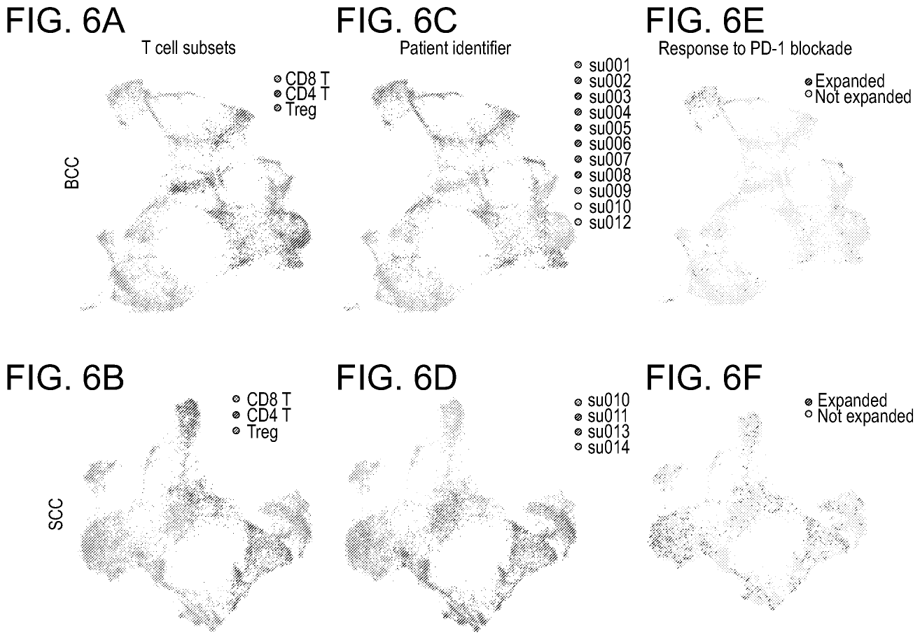

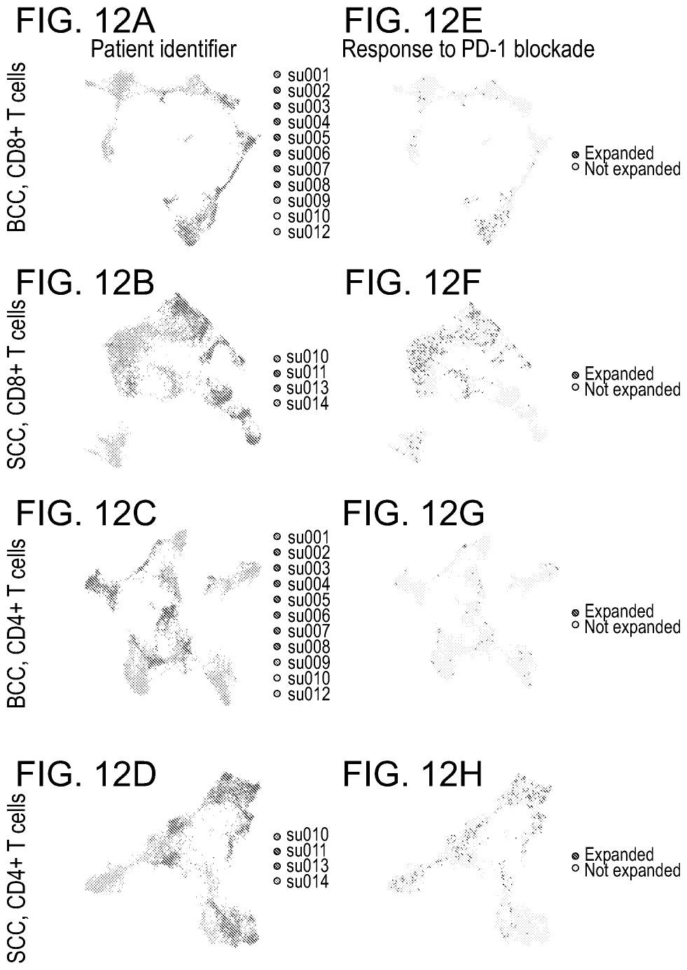

To investigate which T cells responded to ICB by clonal expansion, data from 14 skin cancer patients each with site-matched tumor biopsies from pre- and post-anti-PD-1 treatment was used. Each biopsy was profiled with single-cell RNA-sequencing (scRNA-seq) for gene expression and single-cell TCR-sequencing (scTCR-seq) for clonotype analysis. Samples were separated from basal cell carcinoma (BCC) and squamous cell carcinoma (SCC) for further analysis. Then, clustering and dimensionality reduction algorithms were used to identify cell populations that share common features based on their gene expression profiles and clusters that separated by both T cell subset and patient-specific identifiers were found. See FIG. 6. Clonal expansion triggered by ICB was quantified by identifying T cell clonotypes which match between the pre- and post-treatment samples to calculate the treatment-induced fold-increase of the clonotype size. See FIG. 6. In the BCC data, 2.02% of all CD8+ T cells, 0.63% of all conventional CD4+ T cells, and 0.99% of all FOXP3+ CD4+ Tregs expanded after ICB, with an average fold-increase of 3.82, 2,75, and 4.54, respectively. While CD8+ T cells predominantly responded in BCC, in SCC there was a broader T cell response with 5.14% of all CD8+ T cells, 5.04% of all CD4+ T cells, and 2.70% of all Tregs expanding after ICB, with an average fold-increase of 4.88, 5.26, and 4.06, respectively. The absolute number of Treg clonotypes that responded to ICB with clonal expansion was limited in both datasets. Therefore, Tregs and conventional CD4+ T cells were combined for subsequent analysis. To increase resolution, clonotype expansion analysis on each major T cell subset was repeated and in BCC, the majority of responding CD8+ T cells came from one patient. See FIG. 12. However, still only 6.74% of single CD8+ T cells of this patient were identified as responding. In the SCC data, the responding cells were more dispersed across patients. In conclusion, both CD8+ and CD4+ T cell clonotypes that expanded in response to ICB based on scTCR-seq clonotype profiling in BCC and SCC cohorts were identified.

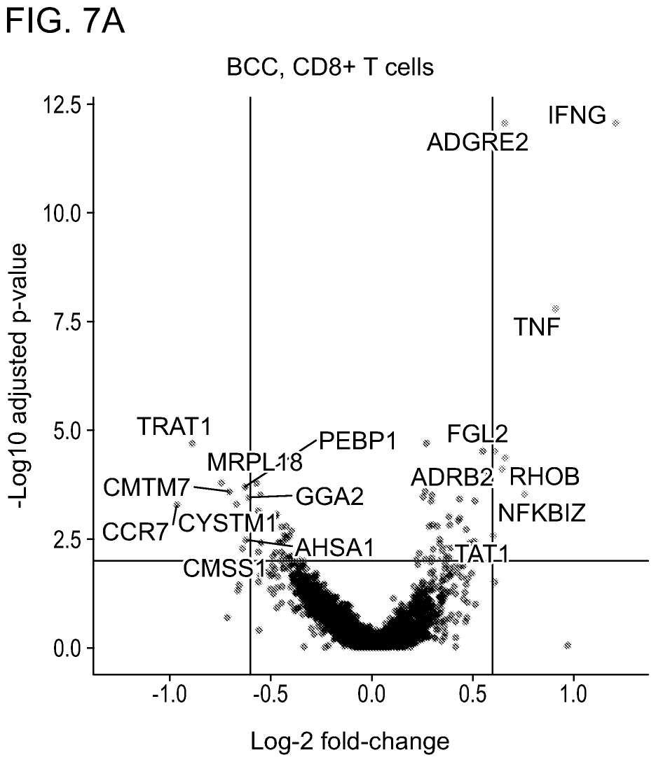

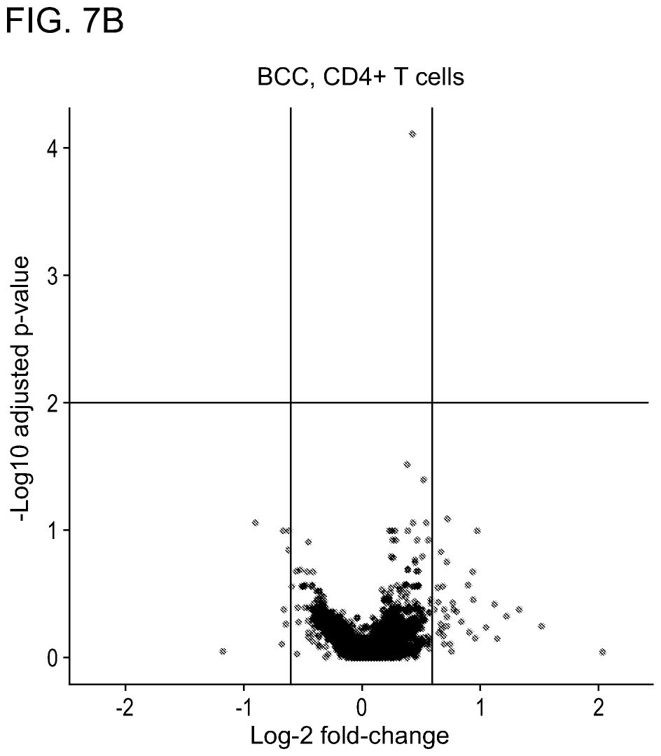

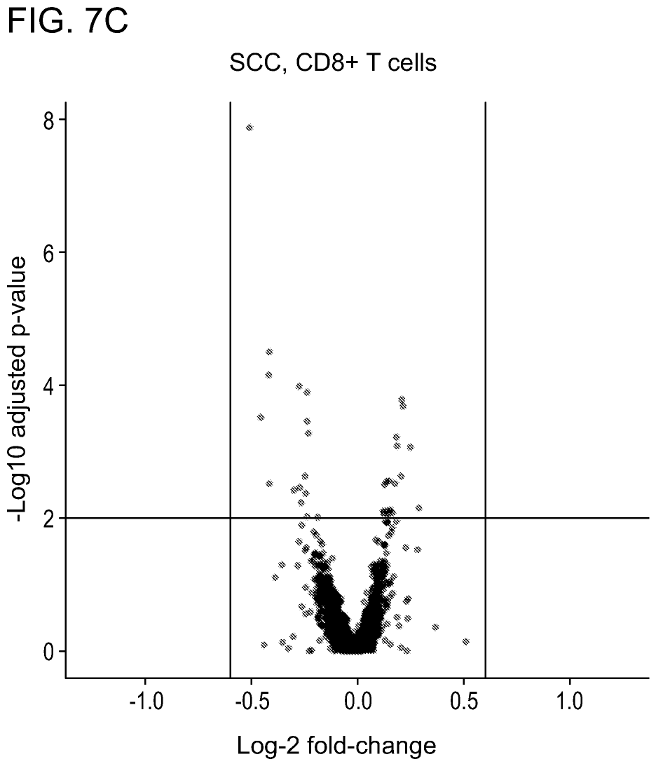

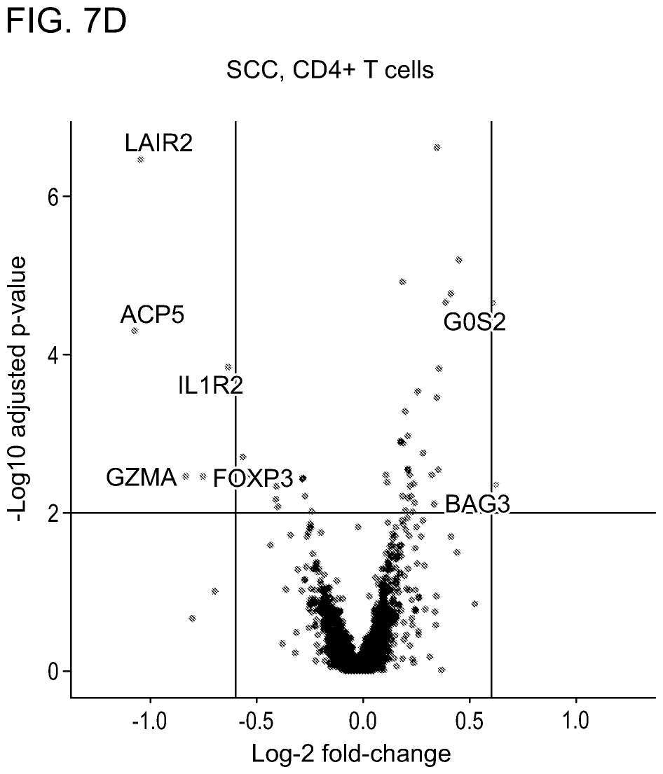

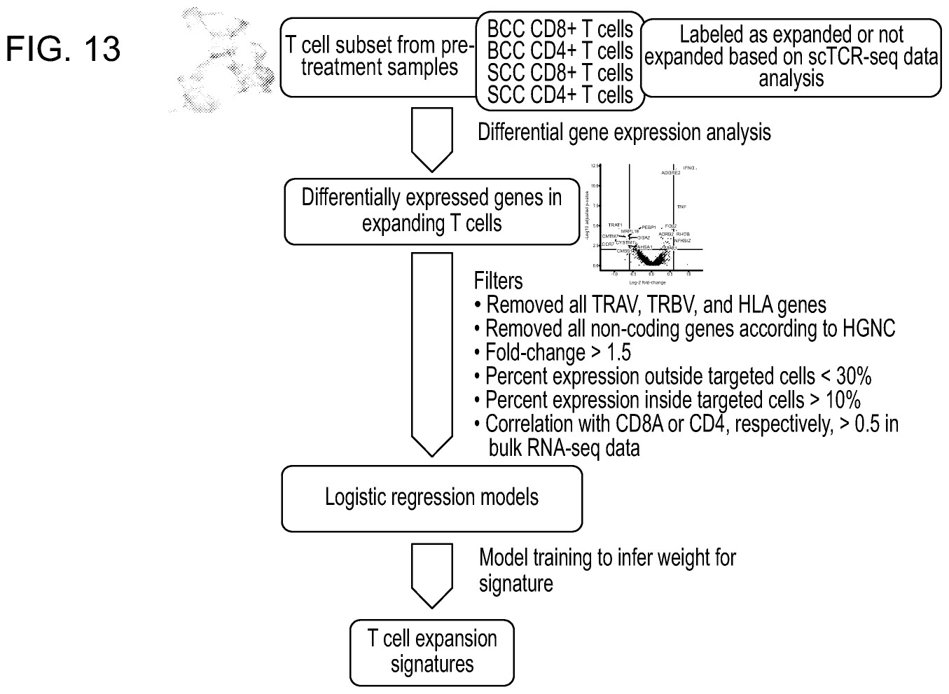

Next, investigating the possibility of using pre-treatment gene expression profiles to distinguish T cells that responded to ICB with clonal expansion from T cells that did not respond was done. Using the CB approach, the TCR clonotypes in the pre-treatment samples were grouped into a fraction that did respond and a fraction that did not respond after ICB. See FIG. 12. Then genes were identified that were differentially expressed in the set of responding cells using their scRNA-seq gene expression profiles. To identify differentially expressed genes, the gene expression fold-change was calculated between the two fractions for each gene and several filtering steps were used to ensure the identified genes represent clear (not affected by sparsity of single-cell data or high baseline expression), meaningful (have an appreciable log fold-change), and statistically robust signals (significant based on multiple-testing-corrected rank-sum test). See FIG. 13. In BCC patient samples, 8 genes (IFNG, ADGRE2, TNF, FGL2, RHOB, ADRB2, NFKBIZ, STAT1) that were upregulated and 9 genes (TRAT1, MRPL18, PEBP1, CMTM7, CCR7, CYSTM1, AHSA1, CMSS1) that were downregulated in CD8+ T cells relative to clonotypes that did not expand following ICB were found. See FIG. 7. In contrast, there were no differentially expressed genes in CD4+ T cells. See FIG. 7. This may be due to the low frequency of expanding cells in this subset (0.7%). In SCC samples, no differentially expressed genes in CD8+ T cells were found, but 5 downregulated (LAIR2, ACP5, IL1R2, GZMA, FOXP3) and two upregulated (G0S2, BAG3) genes in CD4+ T cells relative to clonotypes that did not expand following ICB were identified. See FIG. 7. In summary, two distinct sets of differentially expressed genes at the single-cell level, one linked to CD8+ T cells in BCC and another linked to CD4+ T cells in SCC, were identified that correlated with the clonal expansion in response to ICB.

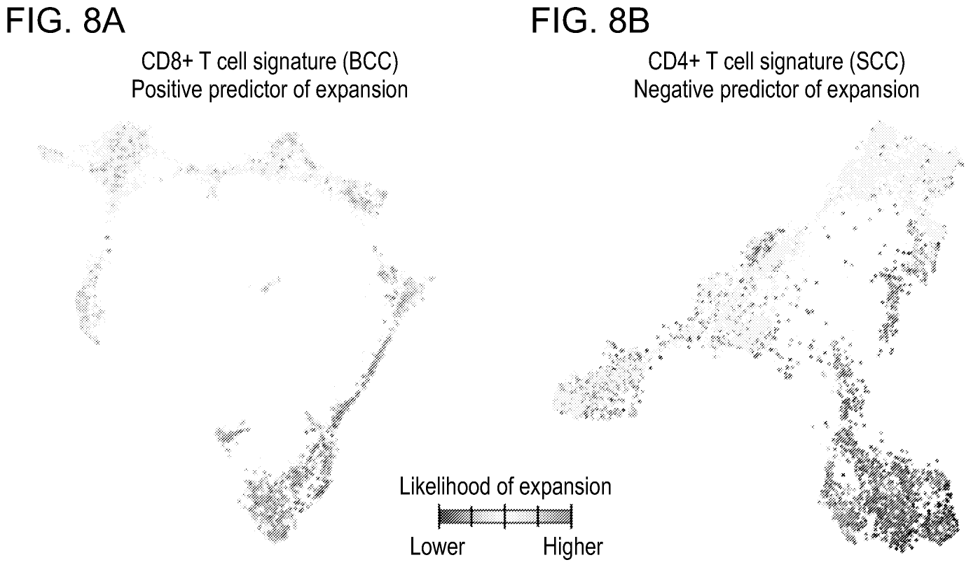

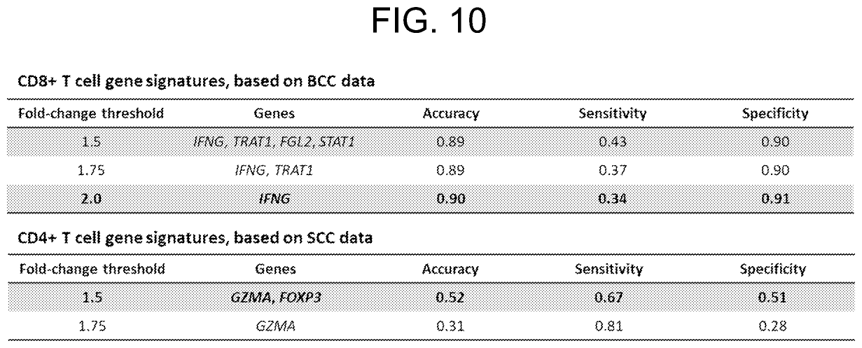

Given the sets of differentially expressed genes in CD4+ and CD8+ T cells, finding which single-cell gene expression signature best predicted the response to ICB that could later be applied to bulk gene expression data to predict patient response to ICB in clinical trials was important. Bulk RNA-seq data not only represents tumor-infiltrating T cells, but the mixture of all cells in the TME, including immune, stromal, and malignant cells. To obtain a T cell-specific predictive signature, the list of differentially expressed genes was restricted to genes with expression more restricted to T cells subsets by computing their correlations with T cell lineage markers. To obtain a gene expression signature that best predicted T cell expansion after ICB at the single-cell level, a logistic regression model on the remaining differentially expressed genes was trained. See FIG. 13. Whether more stringent cut-offs for differential expression, which lead to decreased numbers of genes in the signature, would impact the predictive performance of the logistic regression models was also investigated. See FIG. 10. For the CD8+ T cell expansion signature, the most parsimonious model with just one gene, IFNG, had the highest accuracy and specificity as a positive predictor of expansion. FIG. 8, c.f. FIG. 12. For the CD4+ T cell signature, a model with two genes, GZMA and FOXP3, was found to have the best accuracy and specificity; CD4+ T cell transcription of both these genes were negative predictors of expansion (i.e. prior to treatment these transcripts were downregulated in clonotypes that did respond versus those that did not respond after ICB). FIG. 8, c.f. FIG. 12.

In conclusion, gene expression signatures that predicted response to ICB at the single-cell level were identified: expression of one signature was a positive predictor of CD8+ T cell expansion (CD8-expansion signature) and, inversely, expression of the other signature was a negative predictor of CD4+ T cell expansion (CD4-nonexpansion signature).

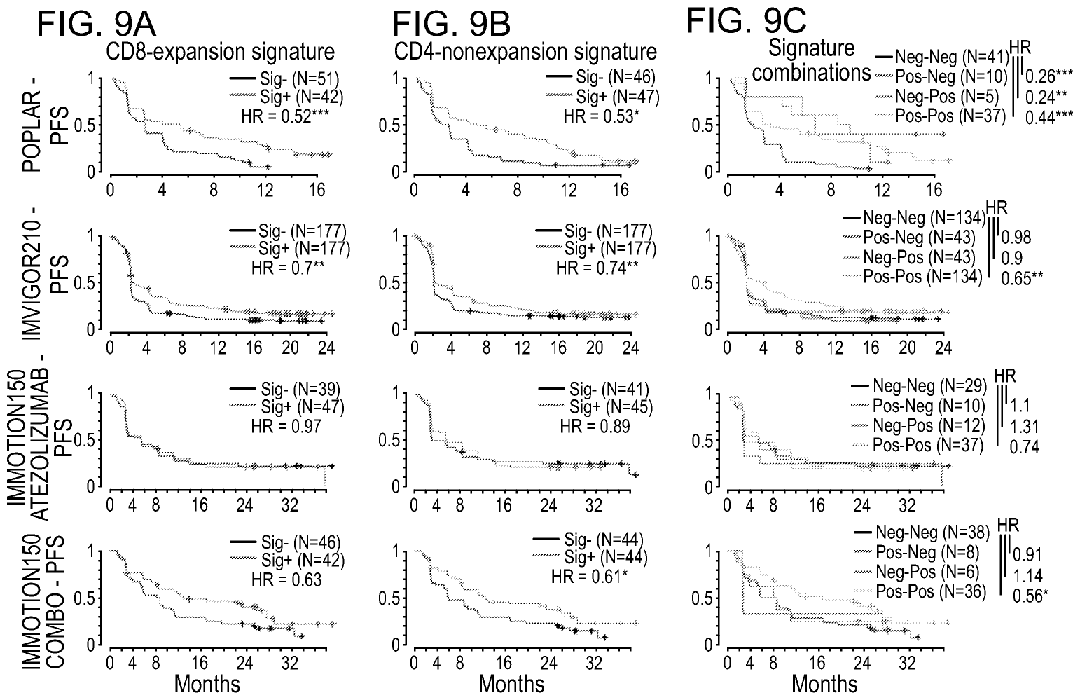

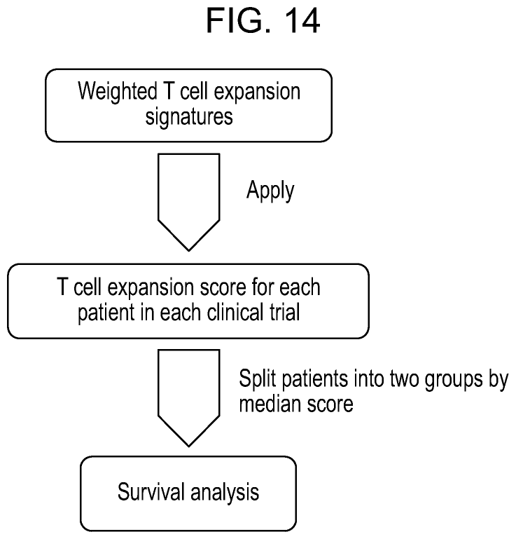

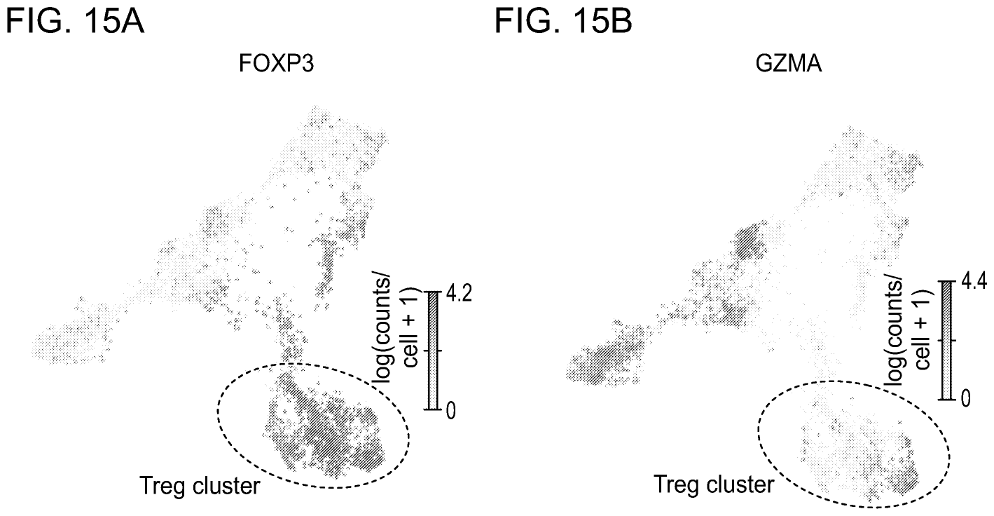

To investigate if the two gene expression signatures derived from single-cell data (FIG. 8) can predict ICB treatment outcome, both the CD8-expansion signature and the CD4-non-expansion signature were applied to data from three phase II clinical trials with an anti-PD-L1 checkpoint inhibitor (atezolizumab) where bulk RNA-seq on pre-treatment patient samples was available: the POPLAR study16 (NCT01903993) in locally advanced or metastatic non-small-cell lung cancer, the IMvigor210 study21 (NCT02951767, NCT02108652) in locally advanced or metastatic urothelial bladder cancer, and the IMmotion150 study22 (NCT01984242) in advanced renal cell carcinoma. For each study, a signature score for each patient was generated, a median cutoff was used to distinguish signature-high from signature-low patients, and investigation was conducted as to whether the signature can predict outcome to treatment using survival analysis. See FIG. 14. For the CD8-expansion signature, signature-high patients trended toward longer progression-free survival (PFS). See FIG. 9. For the CD4-non-expansion signature, signature-high patients also tended to experience longer PFS. See FIG. 9. This was surprising because a high signature means that CD4+ T cells in their tumors would have a lower probability to expand post ICB. Expression of FOXP3 and GZMA, the genes contributing to this signature, was found within distinct conventional CD4+ T cell clusters, however, there was also some overlap in the Treg cluster. See FIG. 15. Therefore, the potential predictive importance of this non-expansion signature originating from within the Treg subset cannot be excluded. While both signatures significantly (p-value<0.05) enriched for responding patients in IMvigor210 and POPLAR, only patients in the IMmotion150 trial with the CD4-nonexpansion signature reached significance in the combination arm (atezolizumab+bevacizumab).

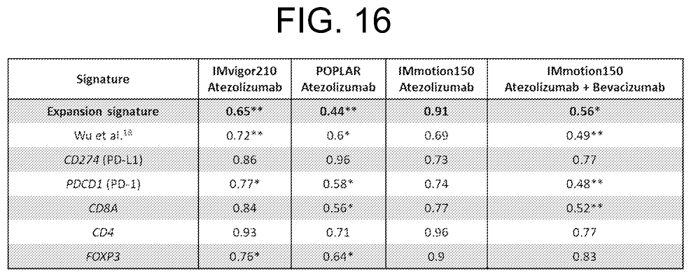

Next, identifying whether applying both signatures in combination can further improve treatment outcome predictions was investigated. The patients were divided into four groups with the CD8 signature indicated first: above median score for both signatures (Pos-Pos), above median score for one signature (Pos-Neg, Neg-Pos), or below median score for both signatures (Neg-Neg). A Pos-Pos combined signature could further improve the predictions for IMvigor210 and the combination arm of IMmotion150. See FIG. 9. For POPLAR, all groups positive for at least one of the two signatures had significantly improved outcome relative to the low-low group. See FIG. 9. Based on these observations, a combined signature that takes both the signatures into account was derived. When compared with other published signatures and gene expression markers of interest including CD274 (PD-L1), PDCD1 (PD-1), CD8A, CD4, and FOXP3, the combined signature consistently achieved similar or superior hazard ratios compared to other signatures or markers of interest. See FIG. 16. Of note, for the IMmotion150 atezolizumab monotherapy arm, none of the signatures was able to provide significantly lowered hazard-ratios (HRs). In summary, gene expression signatures derived from pre-treatment scRNA-seq data from a small number of patients can predict outcome in larger cohorts of ICB treated patients when applied to pre-treatment bulk RNA-seq data. Furthermore, a combination signature can lead to further improved predictions depending on the tumor type.

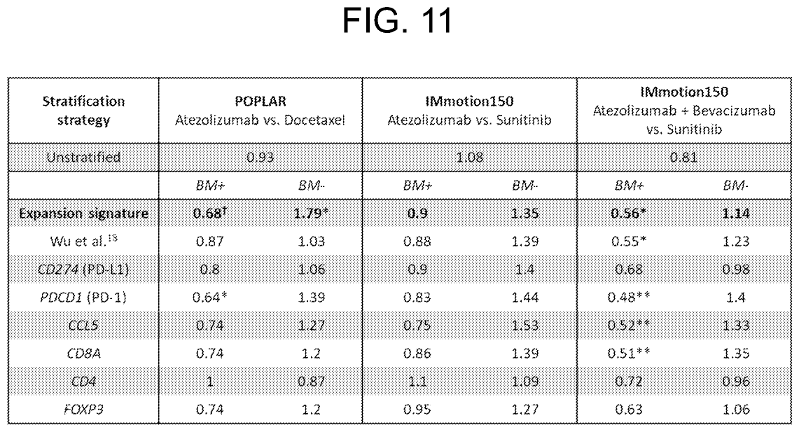

To further demonstrate how predictive signatures derived from single-cell data could impact the clinical development of novel experimental drugs, patient stratification using our combination signature was analyzed to determine whether it could improve the probability of obtaining a significant improved PFS in a clinical trial. For this a subset of patient data of the POPLAR and IMmotion150 studies in which pre-treatment bulk RNA-seq data were available for the experimental and standard-of-care comparator arms was used. The combination signature was used to divide patients treated with ICB in each trial into biomarker-positive (BM+) and biomarker-negative (BM−) groups. For each trial and biomarker group, a survival analysis between the comparator arms and the experimental arms was performed and the results to the outcome in the unstratified patient population were compared. See FIG. 11. In the unstratified case, the experimental arms did not significantly improve HRs in any of the three analyses. After stratifying patients based on the combination signature, overall lowered HRs in the BM+ groups as compared to unstratified. Statistical significance was reached for the IMmotion150 trial combination arm, suggesting that the combination of atezolizumab and bevacizumab is more beneficial than the multi-targeted receptor tyrosine kinase (RTK) inhibitor sunitinib in this patient population. Conversely, in the BM− groups increased HRs were found as compared to unstratified. Significance was reached in the BM− group of POPLAR, suggesting that atezolizumab is less beneficial than the chemotherapy docetaxel in this patient group. Correspondingly, in the BM+ group of POPLAR there was a strong trend towards decreased HR as compared to unstratified; however, it did not formally reach significance (p=0.06). We also compared our combination signature to previously published signatures and other biomarkers of interest. See FIG. 11. The signature consistently showed better or similar performance to other markers of relevance. Of note, for the IMmotion150 atezolizumab monotherapy arm, none of the signatures was able to provide significant lowered HRs. In summary, it was demonstrated that gene expression signatures derived from single-cell data from a small number of patients could prospectively identify populations of patients that have a greater chance of benefiting from an experimental treatment compared to patients in an unstratified patient population.

Using pre- and post-treatment single-cell data from patients who received ICB, this example demonstrated a novel approach to identify predictive gene expression signatures that can be applied as biomarkers that stratify patients to improve the outcome of clinical trials. The herein described clonotype barcoding (CB) approach uses the TCR sequence intrinsic to each T cell to match clonotypes between pre- and post-treatment tumor samples to identify T cells that expanded in response to treatment. The CB approach prioritizes gene expression patterns from expanded and recurring T cell clonotypes detected in the TME for predicting treatment outcome. The CB approach could also be applied to identify predictive biomarkers for therapeutics targeting pathogenic B cells in autoimmune diseases.

Single-cell profiling enables the CB approach to exploit heterogeneity at the single-cell level to derive predictive gene expression signatures. Compared to conventional approaches that require larger numbers of patient samples in order to compare different groups of patients, the ability to link groups of T cells from pre- and post-treatment samples from the same patients provides an elegant way to focus the analysis and identify the most critical predictive parameters. Consequently, this approach allows one to perform an unbiased search for a predictive signature from smaller patient cohorts, such as those available from an early clinical study. The signatures found using the CB approach were surprisingly simple and amenable to interpretation and practical clinical implementation (i.e. immunohistochemistry or in situ hybridization) but would have been missed otherwise as part of more complex signatures. While the single-cell data used in this study was still limited in the number of matching T cell clonotypes pre- and post- treatment, the generation of scRNA-seq datasets with larger numbers of profiled cells will yield more refined signatures that more clearly connects T cell biology to patient responses to immunotherapies. Furthermore, more optimized, and standardized single cell data generation and analysis will enrich the data set further and provide more meaningful analysis.

Mechanistically, the identification of IFNG as the best predictor of CD8+ T cell clonal expansion in BCC patients treated with ICB indicates the presence of functional tumor-reactive cells actively recognizing cognate tumor antigens but whose expansion was inhibited by the PD-1/PD-L1 pathway. It was therefore surprising that a CD8+ T cell signature in SCC patients was not identified, in spite of a greater proportion of pre-treatment CD8+ T cell clones expanding (2.02% and 5.14% of CD8+ T cell clones in BCC and SCC, respectively). T cell clonal responses in SCC were broader than in BCC, with a greater proportion of conventional CD4+ T cells and Tregs expanding in SCC, which may indicate distinct ICB mechanisms of action between these cancer indications. Whereas CD8+ T cell expansion in BCC may have been a direct consequence of ICB, it may have been an indirect consequence in SCC, potentially resulting from CD4+ T cell help , decreased regulatory T cell inhibition, or from anti-tumor activity from immune cell subsets not captured in our CB approach, such as NK cells.

Interpretation of the CD4-non-expansion signature is perhaps more complex. FOXP3 and GZMA do not necessarily correspond to the same cell subset, and GZMA by itself still had predictive power outside of the Treg cluster. While the transcription factor FOXP3 is characteristic of Tregs, FOXP3 transcription is also expressed in conventional T cells following activation, and indeed FOXP3 was found within the conventional CD4 T cell cluster in the SCC scRNA-seq data. GZMA, which encodes the cytolytic protein granzyme A and is associated with effector T cells, has been previously identified as a positive prognosticator or a predictive biomarker for ICB treatment, although this is typically attributed to CD8+ T cells. However, since there is some overlap between FOXP3 and GZMA within the Treg cluster, this Treg subset may have potential predictive importance. Granzyme A produced by activated Tregs reportedly makes these cells more susceptible to self-inflicted apoptosis.

Further, it was demonstrated that the predictive gene expression signatures identified using the CB approach can be validated in larger clinical trials. Both the CD8-expansion signature as well as the CD4-non-expansion signature can predict favorable outcomes to treatment and that a combination of both signatures can lead to further improved results. It is interesting to note that the predictive signatures were derived based on data from skin cancer patients, but that they still showed predictiveness in larger cohorts of three other solid cancer types. However, the best combination of signatures depended on the treated patient population.

Finally, it was demonstrated how the predictive signatures could be applied for patient stratification to improve the probability of success of clinical trials. While the signatures consistently performed better or similarly to other relevant markers, PD-1 expression alone was also a strong predictive biomarker. This is not unexpected based on the mechanistic understanding of ICB. Importantly, the strength of the CB approach was that it could identify a biomarker signature in an unbiased way that performed similarly well. This could be most relevant for identifying biomarkers for novel experimental therapies where the mechanism may be less understood or more complex. Thus, the proposed CB approach is a promising novel use of single-cell data for biomarker identification that will impact future clinical developments of novel immunomodulatory therapeutics.

### Methods

Identification of Clonal Expansion Based on scTCR-seq Data

TCR clonotype labels for individual T cells were obtained from Yost et al. Clonotypes shared by pre- and post-treatment T cells within the same patient were identified based on their matching TCR CDR3 sequences. Clonotypes with post-treatment clonotype size (number of T cells sharing the same TCR CDR3 sequences) greater than the pre-treatment clonotype size were considered to have expanded in response to ICB. For patient su013 in the SCC dataset, 4488 T cells were profiled in the pre-treatment sample but only 69 in the post-treatment sample; this imbalance in T cell counts partially explains why no clonotypes in patient su013 were considered to have experienced expansion after treatment.

Clustering and Dimensionality Reduction of scRNA-seq Data

Clustering of scRNA-seq data from all BCC cell types was performed as previously described with Seurat (version 3.1.1). Based on this first round of clustering and the expression of canonical T cell markers (CD8A, CD4, FOXP3), we identified T cell clusters. The T cells specifically from pre-treatment samples were extracted from the complete dataset then re-clustered. In this round of clustering, the default variable gene selection criteria in Seurat was used. These variable genes were used for principal component analysis (PCA). The first 20 principal components were used for shared nearest neighbor-based clustering with k=20 and resolution=3. The same principal components were used for visualization with UMAP projections with minimum distance=0.1 and number of neighbors=20. The SCC scRNA-seq dataset was already specific to T cells. After extracting the pre-treatment T cells only from this dataset, clustering was performed with Seurat. These pre-treatment T cells were clustered the same way as BCC pre-treatment T cells. After the pre-treatment T cells were clustered, each cluster was annotated as CD8+ T cells, conventional CD4+ T cells, or Tregs based on expression of CD8A, CD4, or FOXP3, respectively.

### Differential Gene Expression Analysis

Using only pre-treatment scRNA-seq data, differential gene expression analysis was performed to determine significantly up- or down-regulated genes in the expanded T cell population versus the non-expanded T cell population. Several filtering steps were taken to remove genes unlikely to be biologically relevant from testing. Testing was limited to genes that were detected in at least 10% of cells in either of the two populations being compared. Non-coding genes, as defined by HGNC, were removed from testing. Human T cell receptor alpha variable (TRAY) genes, human T cell receptor beta variable (TRBV) genes, and human leukocyte antigen (HLA) genes were also removed from testing. For the genes that passed filtering, the Wilcoxon Rank-Sum Test was performed to detect differential gene expression between the expanded T cell population versus the non-expanded T cell population. The Benjamini-Hochberg correction was used to adjust the resulting p-values to control the false discovery rate. Differentially expressed genes were defined to be those with a log2-fold-change >0.6 and an adjusted p-value <0.01. In addition, for genes to be considered significantly upregulated in the expanded T cell population, they were required to be expressed in less than 30% of the non-expanded T cell population. Likewise, for genes to be considered significantly downregulated in the expanded T cell population, they were required to be expressed in less than 30% of the expanded T cell population.

### Generation of Predictive Signatures Based on Differentially Expressed Genes

Differential gene expression analyses yielded two lists of differentially expressed genes: one from the BCC CD8+ T cell dataset and the other from the SCC CD4+ T cell dataset. For each list, genes with a fold-change >1.5 were used as predictors in a logistic regression classifier that predicted the expansion status of each cell. The classifier computed a coefficient for each gene that reflected how strongly the gene expression level of each gene predicted the expansion status of each cell (expanded vs. non-expanded). A positive coefficient indicated that a cell expressing that gene was more likely to be expanded. A negative coefficient indicated that a cell expressing that gene was more likely to be non-expanded. Then the process was repeated with a fold-change threshold of 1.75, then again with a fold-change threshold of 2.0; with increasing fold-change thresholds, the number of genes being used in the logistic regression classifier decreased. The coefficients, accuracy, sensitivity, and specificity of each logistic regression classifier at each fold-change threshold was recorded. For the BCC CD8+ T cell dataset, we compared the performance of the logistic regression classifiers and chose the most parsimonious model that did not suffer from a substantial drop in accuracy, sensitivity, and specificity. The top-performing classifier in this case contained one gene, IFNG, which has a positive coefficient. This was defined as the CD8-expansion signature. Using the same classifier selection criteria for the SCC CD4+ T cell dataset, we defined the CD4-nonexpansion signature as containing two genes, GZMA and FOXP3, each having a negative coefficient. For each gene in an expansion signature, the absolute value of its logistic regression coefficient was taken to be its weight.

### Prediction of Patient Response to ICB by Application of Single-Cell-Based Gene Expression Signatures to Bulk RNA-seq Data

Bulk tumor RNA-seq data from three ICB clinical trials were used to assess the effectiveness of the CD8-expansion signature, the CD4-nonexpansion signature, and those two signatures combined in predicting patient response. In POPLAR (NCT01903993), patients with locally advanced or metastatic non-small-cell lung cancer were treated with atezolizumab (n=93) or docetaxel (n=100). In IMvigor210 (NCT02951767, NCT02108652), patients with locally advanced or metastatic urothelial bladder cancer (n=354) were treated with atezolizumab. In IMmotion150 (NCT01984242), patients with advanced renal cell carcinoma were treated with atezolizumab alone (n=86) or with both atezolizumab and bevacizumab (n=88). For patients in each clinical trial, the weighted sums of the genes in a signature (CD8+ T cell expansion signature or CD4+ T cell expansion signature) were calculated to be the signature score. The median signature score was determined for each clinical trial, and patients who scored above that median were placed in the biomarker-positive group, while the remaining patients in the same trial were placed in the biomarker-negative group. Survival analyses comparing the biomarker-positive and the biomarker-negative groups in each clinical trial arm were performed as previously described by Wu et al. A Cox proportional-hazards model was fitted to the survival data in each arm of each clinical trial; the resulting hazard ratio and p-value of the Wald statistic were reported. The results of these survival analyses were represented in Kaplan-Meier plots. To study the interaction between the CD8+ and CD4+ T cell expansion signatures within a clinical trial, we assigned the patients in each arm to one of four groups: above median score for both signatures (positive-positive), above median score for one signature (positive-negative, negative-positive), or below median score for both signatures (negative-negative). Survival analysis and Kaplan-Meier plotting were performed as described above, with the negative-negative group serving as the baseline comparator to each of the other three groups. As references, CD274 (PD-L1), PDCD1 (PD-1), CD8A, CD4 and FOXP3 were each treated as its own individual unweighted biomarker. Survival analysis comparing the biomarker-positive and the biomarker-negative groups in each arm of each clinical trial was performed as described above.

Prediction of Trial Outcome (Experimental vs. Comparator Arms) via Predictive Signatures

To compare the effectiveness of these T cell expansion signatures between the experimental (atezolizumab, atezolizumab+bevacizumab) and comparator (sunitinib) arms in IMmotion150, the CD8+ and CD4+ T cell expansion signatures were merged into a single signature, with the respective weights of each gene carrying over to the merged signature. First, to establish a baseline, survival analysis comparing each experimental arm with the comparator arm was done to detect any pre-stratification differences in survival. Next, the patients from IMmotion150 were stratified into a biomarker-positive group and a biomarker-negative group based on the merged signature. For each of the biomarker-stratified groups, survival analysis comparing atezolizumab-treated vs docetaxel-treated patients was performed as described above. For POPLAR, the same process was used for survival analysis to detect the effectiveness of these T cell expansion signatures between the experimental and comparator arms. The only difference is that instead of merging the CD8+ and CD4+ T cell expansion signatures, they were kept separate; the patients with an above-median score for either signature (positive-positive, positive-negative, negative-positive) were considered the biomarker-positive group, and the remaining patients were considered the biomarker-negative group. This stratification strategy corresponded to a previous observation that patients in POPLAR who were above-median for either expansion signature had more similar survival patterns with one another than with patients in the negative-negative group. To compare the effectiveness of other signatures between the experimental and comparator arms in IMmotion150 and in POPLAR, the 19-gene dual expansion signature from Wu et al. was applied, unweighted, as a single signature, while CD274 (PD-L1), PDCD1 (PD-1), CD8A, CD4 and FOXP3 were each treated as its own individual unweighted signature.

### REFERENCES

1. Topalian, S. L. et al. Safety, Activity, and Immune Correlates of Anti—PD-1 Antibody in Cancer. New Engl J Medicine 366, 2443-2454 (2012).
2. Rizvi, N. A. et al. Mutational landscape determines sensitivity to PD-1 blockade in non-small cell lung cancer. Science 348, 124-128 (2015).
3. Rooney, M. S., Shukla, S. A., Wu, C. J., Getz, G. & Hacohen, N. Molecular and Genetic Properties of Tumors Associated with Local Immune Cytolytic Activity. Cell 160, 48-61 (2015).
4. Miao, D. et al. Genomic correlates of response to immune checkpoint blockade in microsatellite-stable solid tumors. Nat Genet 50, 1271-1281 (2018).
5. Kümpers, C. et al. Immune Cell Infiltration of the Primary Tumor, Not PD-L1 Status, Is Associated With Improved Response to Checkpoint Inhibition in Metastatic Melanoma. Frontiers Medicine 6, 27 (2019).
6. Kansy, B. A. et al. PD-1 status in CD8+ T cells associates with survival and anti-PD-1 therapeutic outcomes in head and neck cancer. Cancer Res 77, canres.3167.2016 (2017).
7. Gros, A. et al. PD-1 identifies the patient-specific CD8+ tumor-reactive repertoire infiltrating human tumors. J Clin Invest 124, 2246-2259 (2014).
8. Kumagai, S. et al. The PD-1 expression balance between effector and regulatory T cells predicts the clinical efficacy of PD-1 blockade therapies. Nat Immunol 1-13 (2020) doi:10.1038/s41590-020-0769-3.
9. Yi, M. et al. Biomarkers for predicting efficacy of PD-1/PD-L1 inhibitors. Mol Cancer 17, 129 (2018).
10. Sharma, P., Hu-Lieskovan, S., Wargo, J. A. & Ribas, A. Primary, Adaptive, and Acquired Resistance to Cancer Immunotherapy. Cell 168, 707-723 (2017).
11. Higgs, B. W. et al. Interferon Gamma Messenger RNA Signature in Tumor Biopsies Predicts Outcomes in Patients with Non-Small-Cell Lung Carcinoma or Urothelial Cancer Treated with Durvalumab. Clin Cancer Res 24, clincanres.3451.2017 (2018).
12. Postel-Vinay, S. et al. Challenges of phase 1 clinical trials evaluating immune checkpoint-targeted antibodies. Ann Oncol 27, 214-224 (2016).
13. Wong, K. M., Capasso, A. & Eckhardt, S. G. The changing landscape of phase I trials in oncology. Nat Rev Clin Oncol 13, 106-117 (2016).
14. Chabner, B. A. Approval After Phase I: Ceritinib Runs the Three-Minute Mile. Oncol 19, 577-578 (2014).
15. Huang, A. C. et al. T-cell invigoration to tumour burden ratio associated with anti-PD-1 response. Nature 545, 60-65 (2017).
16. Fehrenbacher, L. et al. Atezolizumab versus docetaxel for patients with previously treated non-small-cell lung cancer (POPLAR): a multicentre, open-label, phase 2 randomised controlled trial. Lancet 387, 1837-1846 (2016).
17. Fairfax, B. P. et al. Peripheral CD8+ T cell characteristics associated with durable responses to immune checkpoint blockade in patients with metastatic melanoma. Nat Med 26, 193-199 (2020).
18. Wu, T. D. et al. Peripheral T cell expansion predicts tumour infiltration and clinical response. Nature (2020) doi:10.1038/s41586-020-2056-8.
19. Sade-Feldman, M. et al. Defining T Cell States Associated with Response to Checkpoint Immunotherapy in Melanoma. Cell 175, 998-1013.e20 (2018).
20. Yost, K. E. et al. Clonal replacement of tumor-specific T cells following PD-1 blockade. Nat Med 25, 1251-1259 (2019).
21. Mariathasan, S. et al. TGFβ attenuates tumour response to PD-L1 blockade by contributing to exclusion of T cells. Nature 554, 544-548 (2018).
22. McDermott, D. F. et al. Clinical activity and molecular correlates of response to atezolizumab alone or in combination with bevacizumab versus sunitinib in renal cell carcinoma. Nat Med 24, 749-757 (2018).
23. Baumeister, S. H., Freeman, G. J., Dranoff, G. & Sharpe, A. H. Coinhibitory Pathways in Immunotherapy for Cancer. Annu Rev Immunol 34, 1-35 (2014).
24. Kreiter, S. et al. Mutant MHC class II epitopes drive therapeutic immune responses to cancer. Nature 520, 692-696 (2015).
25. Alspach, E. et al. MHC-II neoantigens shape tumour immunity and response to immunotherapy. Nature 574, 696-701 (2019).
26. Francisco, L. M. et al. PD-L1 regulates the development, maintenance, and function of induced regulatory T cells. J Exp Medicine 206, 3015-3029 (2009).
27. Zappasodi, R. et al. Non-conventional Inhibitory CD4+Foxp3-PD-1hi T Cells as a Biomarker of Immune Checkpoint Blockade Activity. Cancer Cell 34, 691 (2018).
28. Pesce, S. et al. PD/1-PD-Ls Checkpoint: Insight on the Potential Role of NK Cells. Front Immunol 10, 1242 (2019).
29. Subrahmanyam, P. B. et al. Distinct predictive biomarker candidates for response to anti-CTLA-4 and anti-PD-1 immunotherapy in melanoma patients. J Immunother Cancer 6, 18 (2018).
30. Morgan, M. E. et al. Expression of FOXP3 mRNA is not confined to CD4+CD25+ T regulatory cells in humans. Hum Immunol 66, 13-20 (2005).
31. Roufas, C. et al. The Expression and Prognostic Impact of Immune Cytolytic Activity-Related Markers in Human Malignancies: A Comprehensive Meta-analysis. Frontiers Oncol 8, 27 (2018).
32. Inoue, H. et al. Intratumoral expression levels of PD-L1, GZMA, and HLA-A along with oligoclonal T cell expansion associate with response to nivolumab in metastatic melanoma. Oncoimmunology 5, 00-00 (2016).
33. Karreci, E. S. et al. Human regulatory T cells undergo self-inflicted damage via granzyme pathways upon activation. Jci Insight 2, e91599 (2017).

1. A method of identifying a predictive response biomarker (PRB) for treating a disease (e.g., a cancer) using a T/B-cell-targeted immunomodulatory therapy, the method comprising the following steps:
(a) in a pre-treatment sample of the disease (e.g., cancer), identifying response-capable T/B cells having TCR (T Cell Receptor)/BCR (B Cell Receptor) clonotypes identical to TCR/BCR clonotypes of clonally expanded T/B cells in a matching post-treatment sample of the disease (e.g., cancer), wherein said clonally expanded T/B cells in the matching post-treatment sample are clonally expanded following the treatment; and,
(b) generating a list of genes upregulated and/or down-regulated in said response-capable T/B cells in the pre-treatment sample, to create the PRB;
(c) optionally, each gene in said PRB is weighed using a logistic regression coefficient obtained by fitting said each gene as predictor with expansion status and/or clinical response status as outcome.

2. The method of claim 1, wherein step (a) comprises:
(1) generating said matching post-treatment sample by contacting an ex vivo culture of an untreated sample of the disease (e.g., cancer) with a T-cell-targeted immunomodulatory compound, under a condition and for a time period sufficient for an immunomodulatory effect of the compound on a T cell population within the ex vivo culture to manifest;
(2) encapsulating individual T cells isolated, purified, or enriched from said ex vivo culture into picoliter droplets for single-cell profiling of a functional property, thereby separating each encapsulated individual T cells into a first pool of responder T cells and a second pool of non-responder T cells based on the presence or absence, respectively, of said functional property;
(3) determining TCR clonotype for each encapsulated individual T cells in the first pool of responder T cells and the second pool of non-responder T cells, thereby identifying TCR clonotypes of the responder T cells as the TCR clonotypes of clonally expanded T cells in said matching post-treatment sample;
(4) encapsulating individual T cells isolated, purified, or enriched from said pre-treatment sample into picoliter droplets to identify said response-capable T cells having TCR clonotypes identical to TCR clonotypes of said clonally expanded T cells in said matching post-treatment sample; or,
(1) obtaining, from a suitable donor, said pre-treatment sample and said matching post-treatment sample of the disease (e.g., cancer), wherein said matching post-treatment sample has been contacted with the compound, under a condition and for a time period sufficient for an immunomodulatory effect of the compound on a T cell population within the post-treatment sample to manifest;
(2) encapsulating individual T cells isolated, purified, or enriched from said post-treatment sample into picoliter droplets for single-cell profiling of a functional property, thereby separating each encapsulated individual T cells into a first pool of responder T cells and a second pool of non-responder T cells based on the presence or absence, respectively, of said functional property;
(3) determining TCR clonotype for each encapsulated individual T cells in the first pool of responder T cells and the second pool of non-responder T cells, thereby identifying TCR clonotypes of the responder T cells as the TCR clonotypes of clonally expanded T cells in said matching post-treatment sample;
(4) encapsulating individual T cells isolated, purified, or enriched from said pre-treatment sample into picoliter droplets to identify said response-capable T cells having TCR clonotypes identical to TCR clonotypes of said clonally expanded T cells in said matching post-treatment sample.

3. The method of claim 2, wherein step (b) comprises:
(5) using single cell RNA sequencing (scRNA-seq) to identify said list of genes upregulated in said response-capable T cells in the pre-treatment sample, wherein each gene in said PRB has a log2-fold change of >0.2, and is expressed in <40% of T cells with TCR clonotypes that are not clonally expanded in the matching post-treatment sample;
optionally, wherein said scRNA-seq is carried out using a gene panel specifically designed for a mechanism of action of the compound, or a pre-determined gene panel designed to assess immuno-modulation.

4. The method of any one of claims 1-3, wherein
(1) said pre-treatment sample is a blood sample; and/or
(2) the disease is a cancer, such as a solid tumor.

5. The method of any one of claims 1-4, wherein said ex vivo culture is:
(1) freshly isolated from a disease tissue;
(2) established from a stored (e.g., a frozen) disease tissue;
(3) a single cell suspension; and/or,
(4) an adherent culture.

6. The method of any one of claims 2-5, wherein:
(1) the time period is about 12-24 hrs, 24-36 hrs, 36-48 hours, up to 3 days, up to 4 day, or up to 5 days; and/or
(2) the condition comprises: one of a series of concentrations of the compound, and/or with or without combination with a second therapeutic agent;
optionally, the second therapeutic agent comprises a cytokine (e.g., IL-2, TNF), TCR stimulation (e.g., by CD3 cross-linking) and/or co-stimulation (e.g., CTLA, B7, CD28 etc).

7. The method of any one of claims 1-6, wherein the compound is an immuno-modulatory antibody.

8. The method of any one of claims 2-7, wherein step (2) is carried out by a microfluidic device.

9. The method of any one of claims 2-8, wherein said individual T cells are isolated, purified, or enriched by:
(1) isolating, purifying, or enriching a specific T cell subset or population (such as CD4⁺ or CD8⁺ T cells); and/or
(2) generally isolating, purifying, or enriching for T cells.

10. The method of any one of claims 2-9, wherein said functional property comprises: IFN-γ secretion and/or upregulation of an activation marker.

11. The method of any one of claims 3-10, wherein said gene panel comprises a gene for proliferation (such as Ki67); a gene for stem-like feature (such as TCF7); a gene for T-cell activation (such as CD25, Granzyme B, Perforin, CD28, TNF, IL2, IFNG, 4-1BB, CD38, CD69, OX-40, GITR, IL4, IL6); a gene for T cell exhaustion (CD39, CTLA-4, EOMES, PD-1, TIGIT, 2B4, LAG-3, T-bet, TIM3, TOX, CD160, IL-10); a gene for migration (such as CD31, CD103, CXCR5, CXCR4, CCR7, CCR3, CCR4, CCR8, CCR5, CXCR3); and a combination thereof; optionally, the gene panel further comprises CD4, CD8, FOXP3, CD62L, CD44, CD127, CD27, IL33R, and/or PTPRC.

12. The method of any one of claims 2-11, wherein in step (2), said function property is
(1) used to physically separating said responder T cells and non-responder T cells using a microfluidic device based on the presence or absence of said functional property; or,
(2) an activation marker or a response signature (either or both of which can be determined by, e.g., single-cell RNA sequencing), and wherein said responder T cells and non-responder T cells are not physically separated but are virtually distinguished based on the expression or lack of expression of said activation marker or response signature.

13. The method of any one of claims 2-12, further comprising profiling the pre-treatment sample and/or the post-treatment sample to characterize the samples for, e.g., cell type composition (via, e.g., flow cytometry) and target expression on T cells.

14. A method for selecting a patient for treatment of a disease (e.g., a cancer) using a T-cell-targeted immunomodulatory compound, said method comprising: obtaining a clinical response score for the patient, by assessing the expression status and/or expression level of genes in a predictive response biomarker (PRB) of the disease (e.g., cancer) obtained by the method of any one of claims 1-13, to determine whether said clinical response score for the patient exceeds a pre-determined threshold clinical response score;
wherein patients having clinical response scores exceeding the threshold score are identified as being beneficial for said treatment and are selected for said treatment, and/or,
wherein patients having clinical response scores below a second threshold score are identified as not being beneficial for said treatment and are not selected for said treatment.

15. A method for treating a patient for a disease (e.g., a cancer) using a T-cell-targeted immunomodulatory compound, said method comprising:
(i) obtaining a clinical response score for the patient, by assessing the expression status and/or expression level of genes in a predictive response biomarker (PRB) of the disease (e.g., cancer) obtained by the method of any one of claims 1-24, to determine whether said clinical response score for the patient exceeds a pre-determined threshold clinical response score; wherein patients having clinical response scores exceeding the threshold score are identified as being beneficial for said treatment and are selected for said treatment; and,
(ii) administering a therapeutically effective amount of the compound to the patient identified in (i) as being beneficial for and being selected for said treatment.
